(19) United States
(12) Patent Application Publication
(10) Pub. No.: US 2007/0071683 A1
(43) Pub. Date: Mar. 29, 2007

(54) Ultrasonic concentration of carrier particles

(75) Inventors: Paul Dayton, Davis, CA (US); Katherine W. Ferrara, Davis, CA (US); Shukui Zhao, Davis, CA (US); Susannah Bloch, Seattle, WA (US); Terry Onichi Matsunaga, Tucson, AZ (US)

Correspondence Address: FENWICK & WEST LLP, SILICON VALLEY CENTER, 801 CALIFORNIA STREET, MOUNTAIN VIEW, CA, 94041, US

(73) Assignee: The Regents of the University of California, Oakland, CA (US)

(21) Appl. No.: 11/356,843

(22) Filed: Feb. 17, 2006

Related U.S. Application Data
(60) Provisional application No. 60/721,319, filed on Sep. 27, 2005.

(51) Int. Cl. A61K 49/22; A61K 31/337; A61K 39/00; A61K 39/395; A61K 48/00

(52) U.S. Cl. 424/9.5; 514/449; 424/178.1; 424/184.1; 514/54; 514/44

(57) ABSTRACT

Methods, compositions, and apparatus for localized delivery of compounds are provided. In certain embodiments, acoustic streaming force is used to direct carrier particles to a target site, mediate particle internalization, and release associate compounds. Ultrasound radiation is preferred as the source for the acoustic streaming force. Also encompassed are embodiments in which targeting and membrane permeability enhancement are combined with imaging of the treatment site.

### CROSS REFERENCE TO RELATED APPLICATIONS

This application claims the benefit of U.S. Provisional Application No. 60/721,319, filed Sep. 27, 2005, the entire disclosure of which is hereby incorporated by reference in its entirety for all purposes.

### STATEMENT REGARDING FEDERALLY SPONSORED RESEARCH OR DEVELOPMENT

The U.S. Government has certain rights in this invention pursuant to Grant No. 1 CA 37118 awarded by the National Institutes of Health (National Cancer Institute).

### BACKGROUND OF THE INVENTION

1. Field of the invention

The invention relates to methods, apparatus and compositions, useful for targeted delivery of compounds. More particularly, the invention relates to use of acoustic streaming for targeted delivery of compounds including therapeutic agents and imaging agents.

2. Description of the Related Art

Ultrasound is used in medical settings as a diagnostic aid for imaging internal structures. Advantages of ultrasound over other imaging forms include low cost, portability, and safety. Ultrasound contrast agents are well known in the prior art. Typically these agents comprise vesicles having diameters on the order of hundreds of nanometers, a liquid core, and an oil, lipid, polymeric, or proteinaceous shell. Ultrasound contrast agents improve contrast by acting as sound wave reflectors due to acoustic differences between the agents and surrounding liquid.

A variety of therapeutic uses of ultrasound also have been described. Some applications take advantage of the ability of high intensity ultrasound waves to generate heat and thus destroy structures such as tumors or blood vessels. Such methods lack specificity and can damage healthy tissue.

By focusing the ultrasound energy at a desired delivery site such as, e.g., a tumor, higher local concentrations of a therapeutic agent may be achieved. Use of acoustically active carriers permits simultaneous visualization of the carrier to aid or confirm diagnosis and localize a treatment site. Coupling diagnostic and therapeutic ultrasound modes provides the additional advantage of allowing a clinician administering treatment to confirm carrier fragmentation at a desired treatment site.

Solid tumors rely on the formation of new blood vessels, i.e., angiogenesis, to establish the blood supply necessary to support tumor volumes in excess of a few cubic millimeters. Neo-vascularized tumors have leaky capillaries as compared to normal tissues. This provides a basis for concentrating agents within tumors by administering the agents in carriers that are too large to extravasate through normal capillaries but not too large to extravasate through leaky capillaries. Ultrasound contrast materials loaded with therapeutic agents have been proposed for this purpose. For example, U.S. Pat. No. 5,558,092 describes compositions, methods and apparatus for carrying out diagnostic and therapeutic ultrasound.

The prior art also teaches improving specificity and reducing toxicity for therapeutic agents by targeting carriers. Targeting may involve ligand-receptor interactions such as, e.g., through a monoclonal antibody or other ligand on the surface of the carrier designed to bind to an antigen expressed at the treatment site, or through charge interactions, or other mechanisms; Such interactions require the carrier and target site to approach to within a few nanometers.

The prior art has taught use of radiation force created by ultrasound energy to manipulate acoustically active carriers such as microbubbles. Such manipulations can be used to bring the carrier to the edge of a blood vessel, or slow the velocity of a carrier within a blood vessel to promote binding of the carrier to a cell or biological matrix. In addition, recent advances have recognized the additional benefits created by use of ultrasonic steering of targeted carriers, such as acoustically active liposomes, engineered for therapeutic (cf. diagnostic) purposes. Such carriers are engineered to be acoustically active, carry compounds such as drug payloads, and optionally to have a targeting moiety. In addition, these recent developments have recognized benefits created by combined use of therapeutic ultrasound to promote tissue permeability (sonoporation) with steering and fragmentation.

However, radiation force depends on the presence of a large acoustic impedance mismatch between the carrier and the fluid into which the carrier is introduced thus restricting the choice of carrier, and targeting requires preparation of specialized carriers that require ligand-receptor interactions, whose performance depends on molecular apposition of the ligand and its cognate receptor. Such apposition may be difficult to achieve in flowing systems. Thus, there is a need in the prior art for methods that permit expanded use of different types of carrier particles, and that enhance the targeting ability of specialized carriers.

The present invention addresses these and other deficiencies of the prior art as described more fully below.

### SUMMARY OF THE INVENTION

The present invention is defined by the following claims, and nothing in this section should be taken as a limitation on those claims. Disclosed herein are methods, compositions, and apparatus for targeted delivery of compounds and carrier particles using acoustic streaming.

Accordingly, one aspect of the invention includes methods of using acoustic streaming to target a carrier particle to a site. In one aspect, the acoustic streaming is generated using ultrasonic radiation. In another aspect, the carrier particle is engineered to carry a compound such as a drug payload. In another aspect, the invention includes methods in which carrier particles are used for imaging. In yet another aspect, particles are internalized, fuse with cell membranes, or extravasate optionally as a result of insonation. Yet other aspects of the invention include methods that combine imaging with the above methods, as well as methods that include administering agents or radiation to affect tissue permeability or otherwise alter cell physiology at the site.

In one embodiment, the carrier particle includes a molecule to further improve targeting. In a preferred embodiment, there exists an acoustic mismatch between the carrier particle and the surrounding tissue or liquid. Exemplary embodiments include carrier particles having a liquid core, but also include in some embodiments particles having solid or gas cores. Carrier particles having a core containing an oil are preferred for targeted delivery of hydrophobic agents, whereas particles having a core containing water or other polar solids would be preferred for targeted delivery of hydrophilic agents.

In a preferred variation of the invention, targeting is accomplished using acoustic streaming to concentrate carrier particles along a vessel wall. In another preferred variation, targeting is accomplished using acoustic streaming to reduce carrier particle velocity within a vessel.

In addition, the invention provides methods of targeted delivery of compounds without carrier particles by altering tissue permeability or cell physiology at a target site by administering agents or radiation to affect tissue permeability or otherwise modulate cell physiology at the site. In preferred embodiments the tissue comprises a vessel or a tumor. In another preferred embodiment, the administered radiation is ultrasonic radiation.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates the system used to examine the translation velocity of insonified nanoparticles according to one embodiment of the present invention.

FIG. 2 illustrates the effect of acoustic streaming in deflecting the path of carrier particles.

FIG. 3 combines typical frame and streak images for a lipid-encapsulated, decafluorobutane-filled microbubble (FIG. 3a) and a 100% perfluorohexane nanoparticle (FIG. 3b).

FIG. 4 is a graph illustrating translation velocity of insonified nanoparticles in microns/second for 10, 5, and 2.25 MHz, at pulse repetition frequencies of 4, 8, 16, and 32 kHz.

FIG. 5 is a graph illustrating translation velocity in microns/second of insonified nanoparticles for 10 MHz (FIG. 5a) and 5 MHz (FIG. 5b), as functions of peak negative pressure and acoustic intensity.

FIG. 6 is a graph illustrating the translation velocity in microns/second for nanoparticles and polystyrene beads insonified at 10 MHz and three acoustic intensities.

FIG. 7 shows fluorescence microscopy images illustrating the buildup of fluorescent material from targeted nanoparticles along the wall of a 200 micron vessel before application of ultrasound (FIG. 7a) and during insonation (FIGS. 7b & 7c).

FIG. 8 is a graph illustrating relative quantitation of the brightness of a phantom vessel through which fluorescent nanoparticles are flowing over 30 second intervals without the application of ultrasound, with ultrasound, and after ultrasound has been removed.

FIG. 9 illustrates fluorescence microscopy of PC3 monolayers exposed to targeted nanoparticles containing DiI and ultrasound treatment at 5 MHz and 2.4 W/cm2 for 2 minutes (FIG. 9a), and no ultrasound (FIG. 9b).

FIG. 10 is a graph illustrating quantitation of brightness of control PC3 cells (cells) and PC3 cells exposed to fluorescent targeted nanoparticles (particles) and ultrasound at 10 kHz and 2.4 W/cm2, with center frequencies varying from 10, 5, 2.25, and 1 MHz.

FIG. 11 is a graph illustrating simulations of translational velocity of perfluorohexane nanoparticles from the radiation force component only at 10 MHz and 480 mW/cm² for varying radius, R₀.

### DETAILED DESCRIPTION OF PREFERRED EMBODIMENTS

Briefly, and as described in more detail below, described herein are methods, compositions and apparatus for improving the efficacy and diminishing the toxicity of administered compounds. The improvements are realized by using acoustic streaming of fluid containing carrier particles, such as that produced by, e.g., ultrasonic radiation, to concentrate carrier particles at target sites, such as along vessel walls, within tumors, in cavities, or at other predetermined sites. Once carrier particles have been concentrated at the target site, the carrier particles optionally may be further insonified to promote fusion with cell membranes, extravasation of carrier particles, or otherwise promote release of a compound associated with the carrier particle. The details of the parameters required to manipulate a carrier particle by acoustic streaming are described further within, and with respect to a model useful for predicting carrier particle behavior.

Several features of the current approach should be noted. Combinations and subcombinations of various approaches involving use of acoustic streaming, alone or in conjunction with radiation force, to affect carrier particle localization or velocity in a targeted and predetermined way, fusion of the carrier particle with the localized delivery area, imaging of the carrier particle or of target sites, application of agents or radiation to affect tissue permeability or physiology all are contemplated to be within the scope of the present invention. In addition, the invention contemplates use of techniques to improve the specificity and reduce the toxicity of compounds by formulation with carrier particles. In preferred aspects of these methods of the invention, ultrasonic radiation is used to modulate vessel permeability in a targeted region, thereby promoting extravasation and absorption of the administered compound. The compounds used in this aspect of the invention may comprise any therapeutic or diagnostic substance including, by way of example but not limitation, small molecules, peptides, nucleic acids, and synthetic and semi-synthetic analogues thereof.

Advantages of this approach are numerous. Among the advantages are improved specificity and reduced toxicity for administered compounds, and improved treatment outcomes for subjects in need of treatment for a wide variety of medical conditions, especially cancers, cardiovascular diseases, and inflammatory disorders such as rheumatoid arthritis and Crohn's disease.

The invention is useful for diagnostic and/or therapeutic applications in which it is beneficial to administer a compound such as, e.g., a physiologically-active compound, with or without a carrier particle for the purpose of diagnosing and/or treating a medical condition.

### Definitions

Terms used in the claims and specification are defined as set forth below unless otherwise specified.

The term “ameliorating” refers to any therapeutically beneficial result in the treatment of a disease state or condition, e.g., a chronic or acute disease state or condition, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to image a region.

The term “therapeutically effective amount” is an amount that is effective to ameliorate a symptom of a disease. A therapeutically effective amount can be a “prophylactically effective amount” as prophylaxis can be considered therapy.

The term “carrier particle” refers to any particle, such as a microparticle or nanoparticle, with or without a liquid core, which can be concentrated with an acoustic streaming or radiation force.

An “aptamer” is a type of synthetic oligonucleotide that can bind to a particular target molecule, such as a protein or metabolite.

The phrase “administering into a vessel” encompasses direct and remote administration (i.e., directly into the vessel and into a vessel that is in fluidic communication with a vessel into which agent has been directly administered).

The term “vasoporation” refers to either a mechanical increase in vascular permeability secondary to insonation with an ultrasound wave or a chemical increase in vascular permeability achieved locally by using an ultrasound wave.

It must be noted that, as used in the specification and the appended claims, the singular forms “a” “an” and “the” include plural referents unless the context clearly dictates otherwise. In addition, ranges recited are intended to be inclusive of the parameters bounding the range unless the context clearly dictates otherwise. For example, a recited range of between one and ten is intended to include one and ten unless the context clearly dictates otherwise.

Statistical differences, or lack thereof, in the data were determined by the 2-sided Student's T-test for unequal variances. Statistical significance was determined by p<0.05, and lack thereof was determined by p>0.05.

Methods and Apparatus of the Invention

Background

Carrier particle drug delivery. Many oncologic drugs are toxic to normal tissues in addition to tumor cell lines. Paclitaxel, a common chemotherapeutic drug, must be solubilized in cremophore because of its low water solubility. This is undesirable as cremophore is also highly toxic. This systemic toxicity makes it desirable to deliver the antitumor agent directly to the affected area. Unger et al., Invest. Radiol. 33(12):886-892 (December 1998) have demonstrated that paclitaxel can be suspended in a drug delivery capsule with an oil shell and that local delivery of paclitaxel can be effective against brain tumors. The mechanism of action of current microcapsule drug delivery vehicles includes injection into the bloodstream, followed by disruption at the site of interest that causes the contents of the capsule (the drug) to be delivered at the site of interest. However, nanoparticles with a similar oil shell provide a larger drug payload and are more stable under pressure and mechanical stress. In addition, the use of nanoparticles allows for extravasation without particle rupture.

Ultrasound Acoustic Streaming and Radiation Force—Sound propagating through a medium produces a force on particles suspended in the medium, and also upon the medium itself. Ultrasound produces a radiation force that is exerted upon objects in a medium with an acoustic impedance different than that of the medium. An example is a nanoparticle in blood, although, as one of ordinary skill will recognize, ultrasound radiation forces also may be generated on non-liquid core carrier particles. When the medium is a liquid, the fluid translation resulting from application of ultrasound is called acoustic streaming. In various embodiments, said fluid is in vessels or cavities.

We have shown the ability of radiation force to concentrate microbubbles in-vitro and in-vivo. Dayton, et al., Ultrasound in Med. & Biol., 25(8):1195-1201(1999). An ultrasound transducer pulsing at 5 MHz center frequency, 10 kHz pulse repetition frequency (“PRF”), and 800 kPa peak pressure, has been shown to concentrate microbubbles against a vessel wall in-vivo, and reduce the velocity of these flowing agents an order of magnitude. In addition, we have shown that the application of radiation to concentrate drug delivery carrier particles and the combined effects of radiation force-induced concentration and carrier fragmentation. See U.S. patent application Ser. No. 10/928,648, entitled “Ultrasonic Concentration of Drug Delivery Capsules,” filed Aug. 26, 2004 by Paul Dayton et al., which is incorporated herein by reference.

Perfluorocarbon nanoparticles are different from microbubbles because they are approximately an order of magnitude smaller, have a substantially smaller impedance mismatch from blood/water due to their liquid rather than gas core, and are not resonant scatters. This impedance mismatch is due to the differences in the density and speed of sound of the fluorocarbon core compared to the surrounding medium. For example, the acoustic impedance of decafluorobutane, a perfluorocarbon used for some microbubble contrast agents, at 20° C. and 1 atm is approximately 1200 times less than water. See Table 1. The acoustic impedance of perfluorohexane, the main component of the nanoparticles described in this study is only about 1.7 times less than water, and about 730 times greater than decafluorobutane. Thus, the radiation force on perfluorocarbon nanoparticles is substantially smaller than on a gas bubble, and in general, the velocity imparted to a perfluorocarbon nanoparticle due to radiation force will be orders of magnitude less than for a gas-filled bubble for the same acoustic pressure. In addition to radiation force, translation of the particle will also be caused by acoustic steaming. At sufficient intensities, acoustic streaming will result in translation of the fluid itself, including the particles within the fluid. Although the application of streaming is described here to concentrate sub-micron sized nanoparticles, since nanoparticles have the greatest potential to extravasate, this effect also is effective in concentrating micron-sized particles.

Vasoporation—The mechanical effects of ultrasound (with and without microbubbles or nanoparticles) to alter the permeability of vessels, termed vasoporation, has now been well established. Application of ultrasound with specific acoustic parameters causes increases in vessel permeability.

Sonoporation—The mechanical effects of ultrasound (with and without microbubbles or nanoparticles) to alter the permeability of cells, termed sonoporation, has now been well established. Application of ultrasound with specific acoustic parameters causes increases in cell permeability. The term sonoporation insufficiently broad to cover the effects of vasoporation.

In one aspect, the invention provides ultrasonic acoustic streaming to enhance effectiveness of carrier particles such as nanodroplets (alternatively referred to in this specification as nanoparticles) and other particles useful as carrier particles in the practice of the invention. Acoustic streaming and optionally radiation force is used to “push” or concentrate carrier particles along the wall of a vessel. In small blood vessels, particles such as cells or carrier particles tend to flow along the center of the vessel, rather than along the sides. By concentrating the carrier particles along the vessel wall, a larger percentage of a carrier particle-associated compound is delivered to or through the endothelium. In one embodiment, particles extravasate through endothelium, e.g., in the case where the endothelium is “leaky” to particles of the diameter range used. Additionally, particles are internalized into cells at the target area by endocytosis or by particle fusion with the cell membrane according to one embodiment, and this process may be enhanced by ultrasound by mechanism such as sonoporation. Also, carrier particle rupture produced by ultrasound or other radiation sources contribute to these effects in one embodiment by releasing particle contents, in the case of a vasoactive substance, or by the mechanical action of particle disruption affecting membrane integrity.

Additionally, the invention encompasses use of acoustic streaming to assist delivery of targeted carrier particles. Targeted carrier particles have an adhesion mechanism incorporated into the capsule wall that is specific for a molecular signature which often is disease specific, expressed on the endothelium. Since available adhesion mechanisms work on the distance of nanometers, it is important to localize the carrier particles along the vessel wall for such adhesion to occur. Acoustic streaming produced perpendicular to or against the direction of flow reduces the velocity of particles flowing in a fluid. Thus, in another aspect the invention uses acoustic streaming to assist targeted carrier particle delivery, since slower moving particles have a greater opportunity to interact with adhesion mechanisms on an endothelial or other surface.

The invention encompasses the use of radiation force along with acoustic streaming to assist localization and/or delivery of carrier particles.

The invention further encompasses use of acoustic streaming in cooperation with ultrasonic imaging, to allow a user to observe the area being treated, and optionally with sonoporation, to increase permeability of cells in the target area. Also within the scope of the present invention is a system specifically designed to deliver carrier particles with ultrasound.

In one aspect, the invention uses ultrasound and a carrier particle to enhance delivery of a drug or other agent at the desired site in the following preferred manners:

1. Ultrasound, e.g., at center frequencies about 0.1-20 MHz, at an acoustic pressure about 100 kPa-20 MPa, a long cycle length (e.g., about >10 cycles and continuous-wave) OR a short cycle length (e.g., about <10 cycle), and high pulse repetition frequency (e.g., about >500 Hz) is used to produce acoustic streaming to concentrate carrier particles. The specific parameters will depend on the choice of carrier particle, as detailed further below, and can be readily determined by ordinarily skilled artisans having the benefit of this disclosure.

2. Ultrasound, e.g., at center frequencies about 0.1-20 MHz, at an acoustic pressure about 100 kPa-20 MPa, a long cycle length (e.g., about >10 cycles and continuous-wave) OR a short cycle length (e.g., about <10 cycle), and high pulse repetition frequency (e.g., about >500 Hz) is used to produce acoustic streaming to alter the translational velocity of carrier particles. Again, the specific parameters chosen depend on the choice of carrier particle, as detailed further below, and can be readily determined by ordinarily skilled artisans having the benefit of this disclosure.

3. Ultrasound, e.g., at center frequencies about 0.1-20 MHz, at an acoustic pressure about 110 kPa-20 MPa, and a long cycle length (e.g., about >10 cycles and continuous-wave) OR a short cycle length (e.g., about <10 cycle) and high pulse repetition frequency (e.g., about >500 Hz) is used to produce acoustic streaming that causes carrier particles to come in contact with a surface where it is desired that the particles adhere. An example would be where the surface is the endothelial layer within a blood vessel. Again, the specific parameters chosen depend on the choice of carrier particle, as detailed further below, and can be readily determined by ordinarily skilled artisans having the benefit of this disclosure.

4. Ultrasound, e.g., at center frequencies and acoustic pressure in combination such that it produces a spatial peak-temporal average intensity (Iₛₚₜₐ) between about 200 mW/cm² and 8 W/cm² is used for the above-stated purposes.

5. The above where the shell of the particle entirely or partially consists of a lipid, a polymer, or a protein.

6. The above where the center of the particle entirely or partially contains a liquid perfluorocarbon, an oil, or a gas.

7. The application of the above for affecting particles designed to be detectable by an imaging system (a contrast agent).

8. The application of the above for affecting particles designed to carry therapeutic substances or drugs (examples of a therapeutic substance or drug might be a chemotherapeutic or vasoactive substance).

9. The application of the above for affecting particles which incorporate an adhesion mechanism designed to bind to ligands expressed at the site of interest. Examples of some adhesion ligands would be antibodies, peptides, proteins, aptamers, DNA, RNA, peptidomimetics.

10. The application of the above for affecting particles designed to disrupt blood clots.

11. The application of the above for affecting particles designed to disrupt the blood brain barrier.

12. The application of the above for treating inflammation.

13. The application of the above for treating tumors.

14. Any combination of the above techniques.

In preferred embodiments of the invention, a subject in need of diagnosis or treatment receives an injection of carrier particles, preferably loaded with a compound. Preferably the subject is mammalian, and more preferably is human. The compound preferably comprises a therapeutic agent such as, e.g., a drug, nucleic acid, or other therapeutic agent. An ultrasound transducer may be simultaneously, or immediately thereafter positioned over the site of delivery such as, e.g., a tumor, or an inflamed joint, or a vascular lesion. The pulse sequence of the ultrasound scanner produces acoustic streaming and optionally exerts a non-trivial radiation force to displace flowing carrier particles to the walls of blood vessels at the desired site.

The mechanical effects of ultrasound (with and without microbubbles or nanoparticles) to alter the permeability of cells and vessels have now been well established. In addition, targeted drug delivery vehicles and carrier particles have been developed and characterized, with a model developed to predict their behavior. An ultrasound system that implements these developments is provided by the present invention to realize the benefits of the methods of the invention.

In one aspect, the invention provides for a system to combine imaging and drug delivery. The system comprises the following components:

1. The system is capable of sweeping imaging frames through a three dimensional volume. The use of ultra-high speed photography is well documented as a method to analyze the effect of acoustic energy on ultrasound contrast agents, J. E. Chomas, P. Dayton, D. May, and K. Ferrara, “Threshold of fragmentation for ultrasonic contrast agents,” J. Biomed. Opt., 6:141-50 (2001); P. A. Dayton, J. S. Allen, and K. W. Ferrara, “The magnitude of radiation force on ultrasound contrast agents,” J. Acoust. Soc. Am., 112:2183-92 (2002); A. Bouakaz et al., “High-speed optical observations of contrast agent destruction,” Ultrasound in Med. & Biol., 31:391-99 (2005) (all of which are incorporated herein by reference for all purposes). Imaging frames preferably consist of typical clinical center frequencies (e.g., about 2-20 MHz), and typical acoustic pressures (e.g., mechanical index or MI<1.9). The radius-time oscillation and translation of an individual microbubble contrast agent can be observed at high frame rates. The techniques applied previously to microbubble contrast agents in U.S. patent application Ser. No. 10/928,648, incorporated herein by reference, are applicable to nanoparticles to assess nanoparticle response to ultrasound. In one embodiment, such a system comprises an Imacon 468 (DRS Hadland, Cupertino, Calif.) high speed camera system coupled to an Olympus IX-70 (Melville, N.Y.) microscope to optically record nanoparticle behavior. In addition, the system may include a 10 MHz spherically focused transducer positioned where the beam focus overlaps the optical focus, so that objects in the optical focus are exposed to peak pressures from the transducer. In an experimental system, nanoparticles manually micro-injected into a 200-micron cellulose tube are constrained by the tube and thus are positioned in the optical focus. A cellulose tube appropriate for this aspect of the system, e.g., a cellulose tube made by Spectum of Rancho Dominguez, Calif., has a wall thickness on the order of 10 microns, is nearly optically transparent and relatively non-echogenic. Nanoparticles appropriately diluted in water provide approximately one micron-sized nanoparticle per optical field of view. Larger droplets, with diameters on the order of 500-1000 nanometers were chosen for high-speed photography studies, since smaller droplets were below the resolution of the optical system. Optical streak images showing the diameter of a droplet over time were recorded during the incidence of the acoustic pulse on the droplets.

2. The system is capable of magnetic resonance imaging according to one embodiment. As discussed by G. M. Lanza et al. (J. Nucl. Cardiol. 2004), perfluorocarbon nanoparticles can serve as magnetic resonance imaging (MRI) contrast agents when gadolinium is incorporated into their lipid shell, G. M. Lanza et al., “Magnetic resonance molecular imaging with nanoparticles,” J. Nucl. Cardiol., 11:733-43 (2004); A. M. Morawski et al., “Targeted nanoparticles for quantitative imaging of sparse molecular epitopes with MRI,” Magn. Reson. Med., 51:480-86 (2004); A. H. Schmieder et al., “Molecular MR imaging of melanoma angiogenesis with alphanubeta3-targeted paramagnetic nanoparticles,” Magn. Reson. Med., 53:621-27 (2005); S. A. Anderson et al., “Magnetic resonance contrast enhancement of neovasculature with alpha(v)beta(3)-targeted nanoparticles,” Magn. Reson. Med., 44:433-39 (2000); P. M. Winter et al., “Molecular imaging of angiogenesis in nascent Vx-2 rabbit tumors using a novel alpha(nu)beta3-targeted nanoparticle and 1.5 tesla magnetic resonance imaging,” Cancer. Res., 63:5838-43 (2003); P. M. Winter et al., “Molecular imaging of angiogenesis in early-stage atherosclerosis with alpha(v)beta3-integrin-targeted nanoparticles,” Circulation, 108:2270-74 (2003); T. Cyrus et al., “Magnetic resonance nanoparticles for cardiovascular molecular imaging and therapy,” Expert Rev. Cardiovasc. Ther., 3:705-15 (2005) (each of which is incorporated herein by reference for all purposes).

As a result, they may be useful for multi-modality imaging studies. Methods of MRI are well-known in the art and thus are not discussed in greater detail herein.

3. The system is capable of ultrasonic imaging according to one embodiment. Ultrasound is used in medical settings as a diagnostic aid for imaging internal structures. Advantages of ultrasound over other imaging forms include low cost, portability, and safety. Ultrasound contrast agents are well known in the prior art. Ultrasound contrast agents improve contrast by acting as sound wave reflectors due to acoustic differences between the agents and surrounding liquid.

4. In addition, the system combines imaging with therapeutic pulses according to one embodiment. These therapeutic pulses can take several forms:

a. For vehicles that include a targeting mechanism such as a ligand or predetermined charge distribution or are susceptible to acoustic streaming and/or radiation force, the therapeutic system has the ability to apply ultrasound to bring the carrier particle ligand or charges into contact with the cells of interest. In one embodiment, the targeting moiety is selected from an antibody, an antibody fragment, an aptamer, a carbohydrate, a polysaccharide, a polypeptide, a peptidomimetic, a nucleic acid, and a small organic molecule. In addition, the polypeptide is a peptidic adhesion ligand according to one embodiment. To accomplish this goal, a moderate intensity (for example 3 W/cm²) acoustic pulse of long pulse length (for example, 10 seconds) is transmitted to each area within the three dimensional volume. The typical center frequency of operation for the therapeutic pulses will be on the order of from about 100 kHz to about 40 MHz, and more preferably from about 1 MHz-20 MHz. In one embodiment, an ultrasound wave at a center frequency and pressure combination is such that it produces a spatial peak-temporal average intensity (Iₛₚₜₐ) between about 200 mW/cm² and 8 W/cm².

b. To further deliver a drug to a region of interest in the extravascular space, a therapeutic sequence that creates “vasoporation” is transmitted while nanoparticles or other compounds fill the vasculature. In this sequence, therapeutic pulses with a center frequency between about 0.1 MHz-5.0 MHz, and more preferably from about 0.75 MHz-1.5 MHz are applied to each region within the therapeutic volume at an intensity from about 0.1 MPa-10.0 MPa, and more preferably from about 0.75 MPa-5 MPa. These therapeutic pulses in one embodiment are interleaved with the imaging pulses. Subsequent to or concurrently with the application of these vasoporation pulses, a drug that extravasates through this altered vasculature is administered, alone, or in association with a carrier particle.

c. To further enhance internalization of the carrier particles or the drug within the carrier particles, a therapeutic sequence which results in “sonoporation” is transmitted while nanoparticles or other compounds fill the vasculature. In this sequence, therapeutic pulses with a center frequency between about 0.1 MHz-5.0 MHz, and more preferably from about 0.75 MHz-1.5 MHz are applied to each region within the therapeutic volume at an intensity from about 0.1 MPa-10.0 MPa, and more preferably from about 0.75 MPa-5 MPa. These therapeutic pulses in one embodiment are interleaved with the imaging pulses. Subsequent to or concurrently with the application of these sonoporation pulses, a drug is administered alone, or in association with a carrier particle.

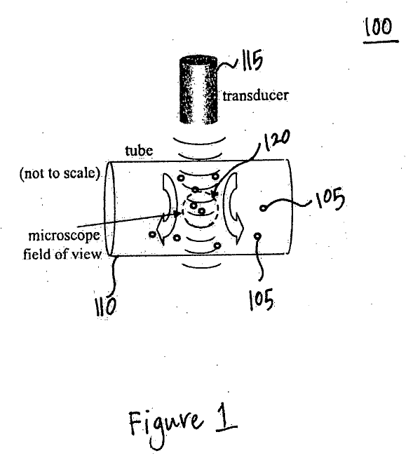

The invention thus contemplates a system to carry out imaging along with therapeutic strategies described in a, b, or c either separately or in combination. FIG. 1 illustrates the system 100 used to examine the translation velocity of insonified nanoparticles 105 according to one embodiment of the present invention. The system 100 shown indicates the orientation of the tube 110, transducer 115, and optical field of view 120, which is represented by the dotted circle. Particle velocity was measured by offline analysis of recorded video frames. An exemplary system provided by the present invention therefore includes the following aspects:

1. Transducer—Ultrasound was produced with either a 10, 5, 2.25, or 1 MHz ¾″ single-element transducer spherically focused at 2″ (IL1006HP, IL0506HP, IL0206HP, IL0106HP; Valpey Fisher, Hopkinton, Mass.) according to one embodiment. Transducer excitation was provided by an arbitrary waveform generator (AWG2021, Tektronix, Irvine, Calif.) and an RF amplifier (3200L, ENI, Rochester, N.Y.). Transducers had bandwidths on the order of 15%-20%. The −6 dB beamwidth at the transducer focus, where samples were placed, was approximately 0.4, 0.8, 1.8, or 4 mm, at 10, 5, 2.25, and 1 MHz, respectively. Acoustic pressure measurements and simultaneous optical and acoustical alignment were performed with a calibrated needle hydrophone (PZT-0400, Onda Corp, Sunnyvale, Calif.).

2. In another embodiment a combined imaging and therapeutic transducer is provided. The transducer uses an interface strategy such as is used for a 1.5 D array with the center array used for imaging and the outer arrays used for the therapeutic pulses. Such an arrangement is described in, e.g., U.S. Pat. No. 5,558,092, the disclosure of which is hereby incorporated by reference in its entirety.

4. The transducer may be scanned mechanically to treat and or image the required three dimensional target site. Scanning may be accomplished manually, or automatically using computer guided robotics, as is well known to ordinarily skilled practitioners.

5. In one embodiment, the ultrasound system timing is adjusted such that both imaging and therapeutic pulse sequences can be transmitted. Further modifications to parameters such as, e.g., the duty cycle, pulse length, acoustic pressure, and center frequency may be altered by the practitioner or system depending on the flow rate of blood vessels at the desired site, the depth of the region of interest, and the specific properties of the carrier particle.

6. Hydrophone—Measurements of translational velocity of nanoparticles in an acoustic field were made using a microscopy system in which acoustical and optical focal volumes were aligned with the needle hydrophone. A 0.7 mm diameter polyester tube (Advanced Polymers, Salem, N.H.) which was nearly optically transparent and weakly echogenic was placed into the mutual acoustical-optical focus, and nanoparticles were injected into the tube using a manual microinjector (Narishige International, NY), and then the flow through the tube was stopped. A 600 frame per second camera (Motioncorder Analyzer, Kodak) recorded the movement of the droplets during repeated insonation. The tube position was adjusted so that the region of observation was at the center of the tube, and the droplet direction was perpendicular to the transducer face.

7. Dye Transfer Experiments—To quantify the relative transfer of a fluorescent dye to cell membranes using the combination of ultrasound and perfluorocarbon nanoparticles, a set of in-vitro dye-transfer experiments were conducted. These experiments utilized custom chambers consisting of a steel frame which holds approximately 1 mL of liquid between two 25 mm cover slips made of Thermanox (Nalge Nunc, Rochester, N.Y.). Targeted nanoparticles were loaded with the fluorescent dye DiI, which fluoresces weakly in water, is well retained in cell membranes, and demonstrates very little cell-to-cell transfer, V. A. Gant et al., “A new method for measuring clustering in suspension between accessory cells and T lymphocytes,” J. Immunol. Methods, 156:179-89 (1992)(incorporated herein by reference for all purposes). Nanoparticles were diluted 25 μL to 1 mL in phosphate buffered saline (PBS) before addition to the chamber. Human prostate carcinoma cells (PC3) were plated on one of the Thermanox cover slips and grown to confluence in an incubator (MCO-17AIC, Sanyo, Bensenville, Ill.). For each experiment, a 1-ml solution of diluted perfluorocarbon nanoparticles solution was injected into a static chamber. The static chamber was mounted in a polycarbonate tank containing an ultrasonic transducer such that the acoustic focus was at the center of the cell monolayer. The tank was filled with distilled water and maintained at 37° C. A 0.5-cm thick block of acoustically-absorbent rubber (Aptflex F28, Precision Acoustics Ltd, UK) was placed in the tank behind the chamber in the rear of the box, to minimize multiple reflections. After insonation, the chamber was removed from the tank and disassembled, and the cover slips were thoroughly rinsed with PBS to remove the majority of free droplets. The cover slips were then examined by fluorescence microscopy.

Compositions Useful for Practicing the Invention

Compositions comprising carrier particles and compounds are especially useful for practice of the present invention. In certain embodiments, the carrier particles have some level of acoustic activity, and the compounds are therapeutically active. Such carrier particles and compounds are well known to those of skill in the art, and may be selected without undue experimentation by skilled practitioners having the benefit of this disclosure. Representative examples of useful compositions are described below.

Compounds can be linked to or dissolved within carrier particle lipid coatings, or deposited in subsurface oil layers, or trapped within the carrier particles themselves.

Carrier particle sizes useful for practice of the present invention will vary depending on the makeup of a carrier particle. Particles on the order of hundreds of nanometers in diameter are preferred in one embodiment. In other embodiments, the technique is applicable to particles in the 1-10 micron diameter range. In one embodiment, the diameter of the carrier particles is less than one micron. In another embodiment, the diameter of the carrier particles is less than 750 nanometers. In yet another embodiment, the diameter of the carrier particles is less than 500 nanometers. Described below is a model that is useful for guiding the skilled practitioner on selecting frequencies, pressures, and other parameters, based on the size and physical properties of the carrier particles. Carrier particle size may be determined using, e.g., a Particle Sizing Systems Model 370 sub-micron particle sizer (Particle Sizing Systems, Santa Barbara, Calif.) or Malvern Zetasizer 5000 (Malvern Instruments, Malvern, Worcestershire, UK).

In one embodiment, carrier particles comprise an oil having a density ranging from 0.7 to 1.7 g/ml at 25° C. at 1 atm. In another embodiment, carrier particles comprise an oil having a density ranging from 0.8 to 1.3 g/ml at 25° C. at 1 atm. Examples of appropriate oils include triacetin, soybean, tocopherol oils, or Cremophor.

Another suitable composition for practicing the invention is the use of perfluorocarbon nanoparticles designed for therapeutic delivery (ImaRx Therapeutics, Tucson, Ariz., USA), which are 0.3 μm±0.12 μm in diameter. These nanoparticles contain a core of liquid perfluorocarbons and a mixture of triacetin and tocopherol oils. The oils serve as a carrier medium for hydrophobic drugs. In one embodiment, such drugs inhibit cell division. In one embodiment, such drugs include the chemotherapeutic paclitaxel. The droplets may be stabilized by a lipid membrane containing dipalmitoylphosphatidylcholine (DPPC), dipalmitoylphosphatidyl-ethanolamine polyethyleneglycol MW-5000 (DPPE PEG₅₀₀₀), and dipalmitoylphosphatidic acid (DPPA), 82:10:8, m:m:m (the total lipid concentration was 0.5 mg/mL or 1 mg/mL). The perfluorocarbon mixture in the droplet core consists of 90% perfluorohexane and 10% perfluoropentane or 100% perfluorohexane, which have nearly identical size distributions.

The associated physical properties are shown in Table 1. In one embodiment, the perfluorocarbon liquid core for use with the methods of the invention has a density between 1.5 and 2.1 g/cm³ at 25° C. at 1 atm. In another embodiment, a perfluorocarbon liquid core for use with the claimed methods has a density between 1.6 and 1.8 g/cm³ at 25° C. at 1 atm. In addition, one embodiment encompasses the use of a perfluorocarbon which undergoes a liquid to a gas phase transition between 25° C. and 42° C. at 1 atm. In another embodiment, the particle comprises a perfluorocarbon which undergoes a liquid to a gas phase transition between −20° C. and 0° C. at 1 atm. In another embodiment, the particle comprises a perfluorocarbon which undergoes a liquid to a gas phase transition between 30° C. and 45° C. at 1 atm. Finally, in another embodiment, the particle comprises a perfluorocarbon which undergoes a liquid to a gas phase transition between 50° C. and 60° C. at 1 atm.

Physical properties of materials studied (from H. G. Vacek V. et al., “Velocity of sound measurements in gaseous per-fluorocarbons and their mixtures,” Fluid Phase Equilibria, 185:305-14 (2001); S. M. Shung K. et al., Principles of Med. Imag., San Diego: Academic Press (1992); J. Rose and B. B. Goldberg, Basic Physics in Diagnostic Ultrasound, Wiley (1978); M. Repacholi et al., Ultrasound: Medical Applications, Biological Effects and Hazard Potential, New York: Plenum Press (1987)), each of which is incorporated herein by reference for all purposes.

TABLE 1

| | | | Viscosity | sound | boiling | |
|---|---|---|---|---|---|---|
| Name (values listed @ | Density | Compressibility | kg/ms | speed, | point, | Impedance |
| 20-25 C. and 1 atm) | kg/m³ | (m²/N) | (cP/1000) | m/s | ° C. | kg/m²s (×10⁻⁶) |
| Air | 1.2 | 7.0 × 10\hat{ }6 | 0.000018 | 340 | −195 | 0.0004 |
| perfluorobutane C₄F₁₀ | 9.9 | 7.0 × 10\hat{ }6 | 0.000014 | 100 | −1.7 | 0.001 |
| perfluorohexane C₆F₁₄ | 1700 | 2.5 × 10\hat{ }9 | 0.00067 | 520 | 56 | 0.88 |
| Water | 1000 | 4.6 × 10\hat{ }10 | 0.001 | 1480 | 100 | 1.48 |

For drug-transfer experiments, a peptide-based bioconjugate designed to target the α₆β₁ receptor was incorporated into the lipid shell of the droplet at a weight percent of ˜5%. For dynamic targeting experiments, the peptide was replaced by biotin and experiments were conducted within an avidin-coated cellulose tube as described below. A carbocyanine dye solution (Vybrant DiI V-22885, Molecular Probes, Eugene, Oreg.) was added to the droplet composition at 1% weight percent to serve as a model drug. For comparison to solid particles, 0.5 micron diameter polystyrene beads were substituted for nanodroplets (Polybead, Polysciences, Warrington, Pa.).

As described above, the present invention also may be practiced with carrier particles comprising targeting moieties designed to assist in the targeting of the carrier particle to a site. Such targeting moieties are well known in the art, and may be selected and incorporated into the carrier particles without undue experimentation by ordinarily skilled practitioners having the benefit of this disclosure. Exemplary teachings in the prior art relating to targeting moieties are provided below. In one embodiment, targeting moieties are selected from an antibody, antibody fragments, aptamers, carbohydrates, polysaccharides, polypeptides, peptidomimetics, nucleic acids, and small organic molecules.

Methods suitable for coupling targeting moieties to carrier particles can be found in Hermanson, “Bioconjugate Techniques,” Academic Press: New York, 1996; and in “Chemistry of Protein Conjugation and Cross-linking” by S. S. Wong, CRC Press, 1993, the entire disclosures of which are hereby incorporated by reference in their entirety for all purposes. Other suitable methods are taught in paragraphs 66 through 130 of U.S. Patent Application Publication U.S. 2002/0102215 A1 to Klaveness et al. Specific coupling methods include, but are not limited to, the use of bifunctional linkers, carbodiimide condensation, disulfide bond formation, and use of a specific binding pair, where one member of the pair is on the targeting agent and the other is on the carrier particle, e.g., a biotin-avidin interaction, see, e.g., Dayton et al., J. Acoust. Soc. Am. 112(5):2183-2192 (November 2002), and internal references 10 through 14 cited in the bibliography (Dayton et al., and internal references 10 through 14 are hereby incorporated by reference in their entirety for all purposes).

The use of charged phospholipids are advantageous in that they contain functional groups such as carboxyl or amino that permit linking of targeting moieties, if desired, by way of linking units.

Suitable compositions for practicing embodiments of the invention using targeted carrier particles include those having an avidin biotin bridge to target an antigen as taught by Lindner and Kaul, Echocardiogrqphy 18(4):329-337 (2001). The initial step comprises administration of a biotinylated monoclonal antibody against the antigen followed by administration of avidin, and then administration of an emulsion of carrier particles containing a biotinylated phospholipid. Avidin forms a bridge between a surface expressing the antigen and biotinylated carrier particles.

Suitable targeting moieties and methods for their attachment to carrier particles also are listed in U.S. Patent Application Publication No. US 2002/0071843 A1 to Li et al., in, e.g., paragraphs 0109 through 0116, and in paragraphs 157 through 159, and in paragraphs 131-145 of U.S. Patent Application Publication U.S. 2002/0102215 to Klaveness, et al. the entire disclosures of which are incorporated by reference in their entirety for all purposes. Other suitable targeting moieties include aptamers, and peptidomimetics.

Multivalent binding can be useful to enhance avidity and reduce “off-rates” so that binding persists long enough to permit imaging at convenient times after delivery of the agent. Polyvalent binding is possible with the use of more than one ligand type per carrier particle, or with mixtures of ligand-carrier particle constructs directed at different targets.

The invention may be practiced using a wide variety of different compounds, including therapeutic compounds having widely varying molecular weights, chemical composition, oil/water partition coefficient, etc. Exemplary compounds and carrier particles include shelled nanoparticles that contain concentrated drug in an oil carrier medium especially useful for packaging hydrophobic drugs. Also contemplated within the scope of useful compounds for practicing the invention are nucleic acids, including mRNA, cDNA, genomic DNA, antisense, and RNAi, any of which may further comprise semi-synthetic backbones or synthetic nucleic acids to modify stability or specificity.

Additional exemplary compounds are listed in U.S. Patent Publication No. 2003/0039613 A1 to Unger et al. (incorporated herein by reference in its entirety for all purposes) in, e.g., paragraphs 0156 through 0172 and include antineoplastic agents, hormones, anti-helmintics, antimalarials, and antituberculosis drugs; biologicals; viral vaccines; aminoglycosides; thyroid agents; cardiovascular products; glucagon; blood products; biological response modifiers; antifungal agents; vitamins; anti-allergic agents; circulatory drugs; metabolic potentiators; antivirals; anti-anginals; anticoagulants; antibiotics; antiinflammatories; antirheumatics; narcotics; opiates; cardiac glycosides; neuromuscular blockers; sedatives; local anesthetics; radioactive particles or ions; monoclonal antibodies; genetic material; and prodrugs.

Pharmaceutical Compositions of the Invention

Methods for treatment of various diseases are also encompassed by the present invention. Said methods of the invention include administering a therapeutically effective amount of a carrier particle and a compound, or, in alternate embodiments, of a compound without a carrier particle. The carrier particles and compounds useful for practicing the invention can be formulated in pharmaceutical compositions. These compositions can comprise, in addition to the compounds and optional carrier particles, a pharmaceutically acceptable excipient, bulking agent, buffer, stabilizer or other materials well known to those skilled in the art. Such materials should be non-toxic and should not interfere with the efficacy of the compound. The precise nature of the carrier particle or other material can depend on the route of administration, e.g. oral, intravenous, cutaneous or subcutaneous, nasal, intramuscular, intraperitoneal routes. In the practice of the invention, preferred administration routes include, e.g., intravascularly, intralymphatically, parenterally, subcutaneously, intramuscularly, intranasally, intrarectally, intraperitoneally, interstitially, into the airways, orally, topically, intratumorly. See, e.g., Unger, et al. U.S. Patent Publication No. US 2003/0039613 A1 at paragraph 0202.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, and Lactated Ringer's Injection. Preservatives, stabilizers, buffers, antioxidants, and/or other additives can be included, as required.

Whether it is a polypeptide, antibody, nucleic acid, small molecule or other pharmaceutically useful compound according to the present invention that is to be given to a subject, administration is preferably in a “therapeutically effective amount” or “prophylactically effective amount” (as the case can be, although prophylaxis can be considered therapy), this being sufficient to show benefit to the subject. The actual amount administered, and rate and time-course of administration, will depend on the nature and severity of the condition being treated. Prescription of treatment, e.g. decisions on dose, timing, etc., is within the responsibility of general practitioners and other medical doctors, and typically takes account of the disorder to be treated, the condition of the subject, the site of delivery, the method of administration and other factors known to practitioners. Examples of the techniques and protocols mentioned above can be found in Remington's Pharmaceutical Sciences, 16th edition, Osol, A. (ed), 1980 (incorporated herein by reference for all purposes).

New research studies have demonstrated that blood-brain barrier (BBB) permeability can be greatly increased for both hydrophilic and hydrophobic drugs through the application of systemic compounds that include bradykinin analogs and P-glycoprotein (P-gp) modulators. The disadvantage of the global increase in BBB permeability produced by these compounds is that the simultaneous application of chemotherapy at the desired concentrations can result in a severe neurotoxicity. The present invention addresses this problem since the drug is concentrated on the luminal surface of the endothelial cells in the desired region, and this increased concentration, especially when combined with, e.g., P-gp modulation will lead locally to increased drug concentration in the tumor and brain-surrounding tumor region. Thus, ultimately, by combining increased BBB permeability with local delivery of a chemotherapeutic to endothelial cells in a regions of interest, the methods of the present invention significantly increase the effectiveness of chemotherapy in brain tumors. The examples below are designed to illustrate ultrasound-enhanced local drug delivery of a hydrophobic drug to the brain.

A composition can be administered alone or in combination with other treatments, either simultaneously or sequentially dependent upon the condition to be treated.

Radiation Sources and Parameters

The relationship between carrier particle translation, center frequency, pressure, pulse length and fundamental or harmonic resonance frequencies of insonified carriers are described in, e.g., Dayton, et al., J. Acoust. Soc. Am. 112 (5):2183-2192 (November 2002) (incorporated herein by reference in its entirety for all purposes.) Further teachings about these relationships are set forth in the Examples below.

Ultrasound systems useful for practicing the invention include the phased system array (HDI c000cv, Advanced Technologies Laboratories) for delivering ultrasound and imaging, the system described in U.S. Pat. No. 5,558,092, to Unger, et al., and may include external application, preferred for skin and other superficial tissues, but for deep structures, application of sonic energy via interstitial probes or intravascular ultrasound catheters may be preferred; and a single-element transducer (IL1006HP, IL0506HP, IL0206HP, IL0106HP; Valpey Fisher, Hopkinton, Mass.) along with an arbitrary waveform generator (AWG2021, Tektronix, Irvine, Calif.), and an RF amplifier (3200L, ENI, Rochester, N.Y.).

The physics governing imaging, fusion, and steering (as by, e.g., acoustic streaming and radiation force) are well understood by ordinarily skilled practitioners having the benefit of this disclosure. For example, it is well known that harmonic emissions may be generated from insonated vesicles (usually at 2× frequency of incident therapeutic ultrasonic waves), and that such harmonic emissions are useful for, e.g., imaging.

The peak resonant frequency can be determined by the ordinarily skilled practitioner either in vivo or in vitro, but preferably in vivo, by exposing the carrier particles to ultrasound, receiving the reflected resonant frequency signals and analyzing the spectrum of signals received to determine the peak, using conventional means. The peak, as so determined, corresponds to the peak resonant frequency (or second harmonic, as it is sometimes termed).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current edition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18^{th} Edition (Easton, Pa.: Mack Publishing Company, 1990); Carey and Sundberg Advanced Organic Chemistry 3^{rd} Ed. (Plenum Press) Vols A and B(1992).

Introduction

Many promising studies indicate that ultrasound-enhanced drug delivery vehicles can be used to locally deliver a drug to a region of interest, with ultrasound imaging used to define the region to be treated and to monitor the inflow of the delivery vehicle. We have developed acoustic streaming and radiation force pulse sequences that deflect a drug delivery vehicle to a vessel wall. Drug delivery vehicles, such a the carrier particles described herein, can be engineered to be manipulated by ultrasonic acoustic streaming and/or radiation force through, e.g., the incorporation of a perfluorocarbon liquid core within a lipid shell and a composition of oils that contain the drug of interest. Ultrasound contrast agents typically have a thin shell composed of lipid, albumin, or polymer.

The lipid-stabilized liquid perfluorocarbon core provides a significant acoustic impedance mismatch with blood and tissue (although much less of a mismatch than a gas), and gives these droplets limited acoustic activity. This liquid core distinguishes liquid-filled nanoparticles (nanodroplets) from solid nanoparticles, which are also being considered as imaging and therapeutic agents, G. Fontana et al., “Solid lipid nanoparticles containing tamoxifen characterization and in vitro antitumoral activity,” Drug Deliv., 12:385-92 (2005); V. P. Zharov et al., “Photothermal nanotherapeutics and nanodiagnostics for selective killing of bacteria targeted with gold nanoparticles,” Biophys. J. (2005)(each of which is incorporated herein by reference for all purposes).

With the addition of an oil, these droplets can solubilize hydrophobic compounds, such as many current chemotherapeutic compounds. Thus, these nanoparticles are more stable under pressure and mechanical stress and are capable of carrying a larger drug payload than microbubbles, however they are also less echogenic.

Although microbubbles fragment at relatively low acoustic pressures (Chomas et al. predict acoustic pressures of 300 kPa at 2.25 MHz will destroy most bubbles less than 3 microns in diameter (Chomas et al. 2001), the droplets described herein, made with 90% or greater perfluorohexane, can be insonified with acoustic pressures on the order of several MPa without a detectable change in properties. Thus, the perfluorocarbon nanoparticles remain intact following the application of acoustic pressure that would destroy microbubbles. Thus, a mode of drug delivery using the methods of the present invention in combination with nanoparticles is fusion of the nanoparticle with a cell membrane.

We have demonstrated that acoustic streaming, which acts on a fluid medium to carry carrier particles, has the unique ability to manipulate and concentrate contrast agents and nanodroplets along the wall of a vessel. Carrier particles localized along the vessel wall travel at a reduced velocity, further increasing the capture efficiency of these vehicles by endothelial cells. A drug delivery vehicle, i.e., a carrier particle, containing, e.g., a liquid perfluorocarbon core can be deflected to a vessel wall, and then fuse with endothelial cell membranes or otherwise deliver its payload through, e.g., pinocytosis or endocytosis, allowing the associated therapeutic to be taken up by endothelial cells or extravasate into the extracellular fluid space. The diameter range of these particles, on the order of hundreds of nanometers (approximately ten-fold smaller than commercially-available microbubble contrast agents), provides superior capability for molecular targeting and extravasation through tumor endothelium, G. Kong et al., “Characterization of the effect of hyperthermia on nanoparticle extravasation from tumor vasculature,” Cancer Res., 61:3027-32 (2001); G. Kong et al., “Hyperthermia enables tumor-specific nanoparticle delivery: effect of particle size,” Cancer Res., 60:4440-45 (2000)(each of which is incorporated herein by reference for all purposes).

In particular, paclitaxel released by these vehicles can cross the lipid phase of the membrane into the endothelial cells.

In Example 1 we develop and evaluate a model for the effect of acoustic streaming and radiation force on carrier particles. In Example 2 we examine nanoparticle oscillation and displacement. In Example 3 we evaluate translation of carrier particles resulting from acoustic streaming and radiation force. In Example 4 we example a flowing assay of localized delivery of carrier particles. In Example 5 acoustically-mediated drug delivery is assessed.

Background and Significance

Local Drug Delivery

With the development of new biologically-active therapeutics, many of which are effective at nanomolar concentrations, there is a tremendous opportunity for improvement in therapeutic efficacy as compared with current cancer therapies. In addition, there is great potential for ultrasound-enhanced drug delivery. One great advantage of this approach is that imaging can be coincident with therapy. For purposes of illustration, we develop drug delivery strategies for the unique setting of drug delivery to a prostate cancer cell monolayer in vitro; however, the techniques of the present invention have application in many areas, including cardiology, inflammation, neurology, and peripheral vascular disease.

Local drug delivery to tumors has been considered for many years with a goal of reducing the toxic effects of some compounds on the surrounding tissue. Paclitaxel can be suspended within oil and locally and effectively delivered, for example using the nanodroplets described herein. Previous researchers have demonstrated that particles in the 200-500 nanometer size regime will extravasate into extracellular spaces, and it is hypothesized that these nanoparticles will have the capacity to permeate the endothelium in leaky vascular spaces such as tumors (Kong et al., 2000; Kong et al. 2001). Preliminary data suggest that encapsulation of paclitaxel can greatly decrease neurotoxicity eliminating the need for Cremophor.

With respect to drug delivery to the brain, some unique aspects of such treatment exist due to the tight endothelial cell junctions and multi-drug resistant properties of the BBB. Many approaches have been considered to increase BBB permeability to drugs and several classes of drugs have been considered for delivery. Drugs such as the lipophilic paclitaxel and the hydrophilic carboplatin had not achieved sufficient concentrations within gliomas to produce an effective response.

Paclitaxel does not typically achieve high concentrations within the brain or tumors in large part because it is rapidly transported back into the blood by the P-glycoprotein (“P-gp”) system. P-glycoprotein is a 170,000 dalton membrane protein that functions as a drug efflux pump, and its overexpression is one of the most consistent alterations in the multi-drug resistance phenotype. It has been demonstrated in P-glycoprotein knockout mice that the penetration of paclitaxel into the brain is markedly increased. Numerous agents have been studied in an effort to overcome P-gp mediated multi-drug resistance, including tamoxifen, Valspodar (PSC 833), verapamil, cyclosporine A, and VX-710. Valspodar is a particularly interesting compound, as it sensitizes cancer cells to chemotherapy through the potentiation of ceramide formation. As ceramide is a second messenger in chemotherapy-induced apoptosis, Valspodar is a particularly attractive target for therapeutic co-administration. Whereas paclitaxel alone did not affect tumor volume, co-administration of paclitaxel (intravenous) and Valspodar (given peroral) reduced tumor volume by 90%. In this study, we use ultrasound-enhanced drug delivery to produce locally increased concentrations of paclitaxel, and evaluate its ability to cross the BBB with and without a P-gp modulator. This greatly increased local concentration further increases efficacy, and reduces systemic toxicity. The methods of the invention are superior to convection-enhanced delivery due to the elimination of the requirement for inter-cerebral administration.

Our approach is substantially different than HIFU (high intensity focused ultrasound) approaches that rely upon high ultrasound pressure or increases in temperature to destroy tissue, and approaches that rely solely upon changes in the permeability of the cell membrane. One objective of the present invention is to locally deliver new therapeutics to the brain using acoustic streaming and radiation force. Given that many new drugs are active at nanomolar concentrations, the local delivery of very small quantities of drugs is expected to have a great impact. Many groups have investigated the change in cell membrane permeability produced by the oscillation of an ultrasound contrast agent. In M. Ward, J. Wu, J. F. Chiu, Ultrasound Med Biol 26(7):1169-1175 (September 2000)(incorporated herein by reference in its entirety for all purposes), it was shown that the contrast agent must be located within a few microns of the cell to change the cell membrane permeability, and therefore a large volume of the contrast agent may be needed to cause a clinically significant response.

Nanodroplets as Contrast Agents and Drug-delivery Vehicles

Perfluorocarbon emulsion nanoparticles are under investigation as ultrasound contrast agents and ultrasonically-enhanced drug delivery vehicles. The diameter range of these particles, on the order of hundreds of nanometers (approximately ten-fold smaller than commercially-available microbubble contrast agents), may provide superior capability for molecular targeting and extravasation through tumor endothelium (Kong et al. 2000; Kong et al. 2001). The lipid-stabilized liquid perfluorocarbon core provides a significant acoustic impedance mismatch with blood and tissue (although much less of a mismatch than a gas), and gives these droplets limited acoustic activity. This liquid core distinguishes liquid-filled nanoparticles (nanodroplets) from solid nanoparticles, which are also being considered as imaging and therapeutic agents (Fontana et al. 2005; Zharov et al. 2005). With the addition of an oil, these droplets can solubilize hydrophobic compounds, such as many current chemotherapeutic compounds. Thus, these nanodroplets are more stable under pressure and mechanical stress and are capable of carrying a larger drug payload than microbubbles, however they are also less echogenic.

Lanza, Wickline, et al. have described the use of perfluorocarbon emulsion nanoparticles as ultrasound contrast agents, G. M. Lanza et al., “Molecular imaging and targeted drug delivery with a novel, ligand-directed paramagnetic nanoparticle technology,” Acad. Radiol., 9(2):S330-31 (2002); S. A. Wickline and G. M. Lanza, “Nanotechnology for molecular imaging and targeted therapy,” Circulation, 107:1092-95 (2003); G. M. Lanza and S. A. Wickline, “Targeted ultrasonic contrast agents for molecular imaging and therapy,” Curr. Probl. Cardiol., 28:625-53 (2003)(each of which is incorporated herein by reference for all purposes), and have developed theoretical models for estimating acoustic reflectivity of different perfluorocarbon nanoparticle formulations, J. N. Marsh et al., “Frequency and concentration dependence of the backscatter coefficient of the ultrasound contrast agent Albunex (R),” J. Acoust. Soc. Am., 104:1654-66 (1998); J. N. Marsh et al., “Improvements in the ultrasonic contrast of targeted perfluorocarbon nanoparticles using an acoustic transmission line model,” IEEE Trans. Ultrason. Ferroelectr. Freq. Control, 49:29-38 (2002); J. N. Marsh et al., “Temperature dependence of acoustic impedance for specific fluorocarbon liquids,” J. Acoust. Soc. Am., 112:2858-62 (2002); C. S. Hall et al., “Temperature dependence of ultrasonic enhancement with a site-targeted contrast agent,” J. Acoust. Soc. Am., 110:1677-84 (2001); C. S. Hall et al., “Time evolution of enhanced ultrasonic reflection using a fibrin-targeted nanoparticulate contrast agent,” J. Acoust. Soc. Am., 108:3049-57 (2000)(each of which is incorporated herein by reference for all purposes. Nanoparticles have low acoustic reflectivity in solution; however, their echogenicity increases when they are deposited in a layer, resulting in a targeted contrast agent which is detectable only when adherent at the target site (Lanza and Wickline 2003). Perfluorocarbon nanoparticles can also serve as MRI contrast agents when gadolinium is incorporated into their lipid shell, so they may be useful for multi-modality imaging studies (Lanza et al. 2004; Morawski et al. 2004; Schmeider et al. 2005; Anderson et al. 2000; Winter et al. 2003; Cyrus et al. 2005). In addition to their application as ultrasound contrast agents, perfluorocarbon nanoparticles have also been used as therapeutic delivery vehicles for drugs that inhibit cell division, (Wickline and Lanza 2003); G. M. Lanza and S. A. Wickline, “Targeted ultrasonic contrast agents for molecular imaging and therapy,” Prog. Cardiovasc. Dis., 44:13-31 (2001); I. V. Larina et al., “Enhancement of drug delivery in tumors by using interaction of nanoparticles with ultrasound radiation,” Technol. Cancer Res. Treat., 4:217-26 (2005)(each of which is incorporated herein by reference for all purposes). Examples of such drugs include doxorubicin, paclitaxel, and other therapeutic agents.

Targeted imaging and drug delivery applications for these particles require that the agent collects preferentially at the target site. Often, the targeting mechanism requires formation of ligand-receptor bonds between the agent and the target cells. Since these bonds form over distances on the order of nanometers, the targeted agent must be in close proximity to the target cell for adhesion and retention to occur. Acoustic radiation force can enhance the efficiency of targeted imaging and drug delivery with microbubble-based agents by deflecting targeted particles to the endothelium and facilitating bond formation, J. J. Rychak et al., “Acoustic radiation force enhances targeted delivery of ultrasound contrast microbubbles: in vitro verification,” IEEE Trans. Ultrason. Ferroelectr. Freq. Control, 52:421-33 (2005); P. Dayton, A. Klibanov, G. Brandenburger, and K. Ferrara, “Acoustic radiation force in vivo: a mechanism to assist targeting of microbubbles,” Ultrasound Med. Biol., 25:1195-201 (1999); M. J. Shortencarier, P. A. Dayton, S. H. Bloch, P. A. Schumann, T. O. Matsunaga, and K. W. Ferrara, “A method for radiation-force localized drug delivery using gas-filled lipospheres,” IEEE Trans. Ultrason. Ferroelectr. Freq. Control, 51:822-31 (2004); S. Zhao, M. Borden, S. H. Bloch, D. Kruse, K. W. Ferrara, and P. A. Dayton, “Radiation-force assisted targeting facilitates ultrasonic molecular imaging,” Mol. Imaging, 3:135-48 (2004)(each of which is incorporated herein by reference for all purposes). Radiation force produced on objects with an acoustic impedance several orders of magnitude different from their surrounding medium, such as microbubbles in blood, can produce rapid translation even at acoustic intensities as low as 10 mW/cm². While radiation forces can also deflect liquid and solid particles, the time-averaged intensity required for such effects is substantially greater than for microbubbles.

In this study, we experimentally assess the feasibility of using ultrasound to enhance local targeting of perfluorocarbon nanoparticles targeted to the cell surface integrin α₆β₁, which is upregulated in prostate cancer. The velocity of translating nanoparticles in an acoustic field is measured and compared with theory describing radiation force effects on droplets, solid beads and bulk fluid. Nightingale et al. have shown that a clinical ultrasound system can produce streaming of bulk fluid in breast cysts with a diameter on the order of millimeters or centimeters, K. R. Nightingale and G. E. Trahey, “A finite element model for simulating acoustic streaming in cystic breast lesions with experimental validation,” IEEE Trans. Ultrason. Ferroelectr. Freq. Control, 47:201-15 (2000). Measurements by Shi et al. indicate that the translational velocity produced by acoustic streaming in millimeter vessels decreases with decreasing vessel diameter, X. Shi et al., “Quantitative investigation of acoustic streaming in blood,” J. Acoust. Soc. Am., 111:1110-21 (2002)(each of which is incorporated herein by reference for all purposes).

For many drug delivery applications, acoustic forces must act within capillaries and therefore the effect of these forces in small vessels must be considered. Here, the magnitude of these effects is examined in small vessels over the range of clinically-relevant ultrasound parameters. As an application of this technique, we demonstrate significant enhancement of the delivery of a fluorescent model drug from targeted nanoparticles to a cell monolayer.

### Example 1

### Effect of Acoustic Streaming and Radiation Force on Carrier Particles

Sound propagating through a medium produces a force on particles suspended in the medium, and also upon the medium itself. When the medium is a liquid, the resulting fluid translation is called acoustic streaming. Acoustic radiation force has been studied in detail since the nineteenth century, V. F. K. Bjerknes, Fields of Force, New York: Columbia University Press (1906), and new applications for this force have arisen with the recent application of bubbles and droplets being considered as ultrasound contrast agents and drug delivery vehicles. The radiation force produced on small spheres in an acoustic field has been discussed in detail. V. N. Alekseev, “Force Produced by the Acoustic Radiation Pressure on a Sphere,” Soviet Physics Acoustics-Ussr, 29:77-81 (1983); A. A. Doinikov, “Acoustic Radiation Pressure on a Rigid Sphere in a Viscous-Fluid,” Proceedings of the Royal Society of London Series a-Mathematical and Physical Sciences, 447:447-466 (1994); A. A. Doinikov, “Radiation Force Due to a Spherical Sound Field on a Rigid Sphere in a Viscous-Fluid,” J. Acoust. Soc. Am., 96:3100-3105 (1994); A. A. Doinikov, “Acoustic radiation force on a spherical particle in a viscous heat-conducting fluid. 1. General formula,” J. Acoust. Soc. Am., 101:713-721 (1997); A. A. Doinikov, “Acoustic radiation force on a bubble: Viscous and thermal effects,” J. Acoust. Soc. Am., 103:143-147 (1998); L. V. King, “On the acoustic radiation pressure on spheres,” Proceedings of the Royal Society of London Series A, 147:212-240 (1934); W. Nyborg, “Radiation Pressure on a Small Rigid Sphere,” J. Acoust. Soc. Am., 42:1762 (1967); K. Yosioka and Y. Kawasima, “Acoustic radiation pressure on a compressible sphere,” Acustica, 5:167-73 (1955) (the entire disclosures of which are hereby incorporated by reference in their entirety for all purposes).

A recent review by Doinikov summarizes much of the development and current state of theory regarding acoustic radiation forces, A. A. Doinikov, “Acoustic radiation forces: Classical theory and recent advances,” Recent Res. Devel. Acoustics, 1:39-67 (2003). The radiation force exerted on a liquid droplet by a traveling wave is described in its simplest form in Eq. 1. For comparison, Eq. 2 presents the force exerted on highly compressible bubbles.

$$\begin{matrix}{F}_{d}^{tr} = \frac{2 {πρ}_{0} {\vert A \vert}^{2} {( {kR}_{0} )}^{6}}{9 {( 2 + {λ}_{p} )}^{2}} \{ {[ 3 - ( 2 + {λ}_{p} ) {λ}_{p} \frac{{c}^{2}}{{\tilde{c}}^{2}} ]}^{2} + 2 {( 1 - {λ}_{p} )}^{2} \} & ( 1 ) \\ {F}_{d}^{tr} = \frac{2 {πρ}_{0} {\vert A \vert}^{2} {( {kR}_{0} )}^{6}}{{( 1 - {ω}_{0}^{2} / {ω}^{2} )}^{2} + {δ}_{rad}^{2}} & ( 2 )\end{matrix}$$

wherein ρ₀ is the fluid density at rest, A is the complex amplitude of the velocity potential of the imposed sound field, and δ_{rad}=kR₀ is the radiation damping constant. The ratio of density of the host fluid to sphere material is given by λₚ, and c and \tilde{c} represent the speed of sound in the fluid medium and the fluid inside the droplet, respectively. In addition, ω is the angular driving frequency and ω₀ is the fundamental resonance frequency of the bubble.

These relations are based on the assumption that the host fluid is ideal, i.e., non-viscous and non heat-conducting. In the limiting case c/\tilde{c}→0, Eq. 1 simplifies to the expression for radiation force on a solid particle, L. V. King (1934); A. A. Doinikov, “Acoustic Radiation Pressure on a Rigid Sphere in a Viscous-Fluid.” With this assumption of an ideal fluid medium, these equations describe the force on the particle due to the sound wave, but ignore forces which are due to acoustic streaming which develops around the particle, and forces on the particle due to the acoustic streaming which would develop in the bulk fluid even if the particle was absent.

A significant discrepancy between theory and experiments has been observed when these assumptions have been applied to estimating radiation forces in traveling waves, A. A. Doinikov (1998). Yosioka and Kawasima expanded Eq. 2 to include the heat loss that occurs in the process of volume oscillations of the bubble, represented by Doinikov as Eq. 3, K. Yosioka and Y. Kawasima, “Acoustic radiation pressure on a compressible sphere,” Acustica, 5:167-73 (1955); A. A. Doinikov, “Acoustic radiation forces: Classical theory and recent advances,” Recent Res. Devel. Acoustics, 1:39-67 (2003)(each of which is incorporated herein by reference for all purposes).

$${F}_{b}^{tr} = \frac{2 {πρ}_{0} {\vert A \vert}^{2} ( {kR}_{0} ) ( {δ}_{rad} + {δ}_{th} )}{{( 1 - {ω}_{0}^{2} / {ω}^{2} )}^{2} + {( {δ}_{rad} - {δ}_{th} )}^{2}} \qquad ( 3 )$$

wherein the thermal damping constant is given as δₜₕ.

For highly compressible microbubbles, dissipation results primarily from thermal and radiation losses; the viscous effects are less significant. However, for small less-compressible droplets, viscous effects should be considered since the viscous force on the particle due to acoustic streaming can be as much as 25%-75% of the total force in a limited set of conditions, J. R. Wu and G. H. Du, “Acoustic Radiation Force on a Small Compressible Sphere in a Focused Beam,” J. Acoust. Soc. Am., 87:997-1003 (1990). Doinikov further developed the theory for droplets to incorporate effects of a viscous heat-conducting fluid, (Doinikov 2003). Since the resulting general-formula is extremely intricate, it is analyzed for the limiting cases of weak or strong dissipation. The case for weak dissipation, where λₛ\tilde{λ}ₛ>>R₀>>δᵥ,\tilde{δ}ᵥ,δₜ,\tilde{δ}ₜ, is presented here as Eq. (4).

$${F}_{d}^{tr} = 2 {πρ}_{0} {\vert A \vert}^{2} {( {kR}_{0} )}^{3} [ {T}_{v} \frac{{δ}_{v}}{{R}_{0}} + {T}_{t} \frac{{δ}_{t}}{{R}_{0}} ] \\ Where {T}_{v} = \frac{3 {( 1 - {λ}_{p} )}^{2} [ \begin{matrix}1 + {λ}_{p} {δ}_{v} / {\tilde{δ}}_{v} + 2 {λ}_{n} {\tilde{δ}}_{v} / {R}_{0} + \\ 2 {( {λ}_{n} { \tilde{δ}}_{v} / {R}_{0} )}^{2} + 2 {λ}_{p} {λ}_{n} {δ}_{v} / {R}_{0}\end{matrix} ]}{{( 2 + {λ}_{p} )}^{2} [ {( 1 + {λ}_{p} {δ}_{v} / {\tilde{δ}}_{v} + {λ}_{n} {\tilde{δ}}_{v} / {R}_{0} )}^{2} + {( {λ}_{n} {δ}_{v} / {R}_{0} )}^{2} ]} and \\ {T}_{t} = \frac{( 1 - {λ}_{k} {\tilde{δ}}_{t}^{2} / {λ}_{α} {δ}_{t}^{2} ) [ γ - 1 - ( \tilde{γ} - 1 ) {λ}_{α} {δ}_{p} {c}^{2} / {\tilde{c}}^{2} ]}{2 ( 1 + {λ}_{k} {\tilde{δ}}_{t} / {δ}_{t} )} \qquad ( 4 )$$

wherein λₛ\tilde{λ} represent the sound wave length in the fluid outside and inside the droplet, respectively, R₀ is the equilibrium radius of the particle, δᵥ,\tilde{δ}ₜ are the viscous penetration depths in the fluid outside and inside the particle, respectively, and δₜ, \tilde{δ}ₜ represent the thermal penetration depth in the fluid outside and inside the particle, respectively. In addition, λ_{α}, λ_{κ}, λ_{π} represent the ratio of volume thermal expansion coefficient of the host fluid to sphere material, the ratio of thermal conductivity of the host fluid to sphere material, and the ratio of dynamic viscosity of the host fluid to sphere material, respectively.

Note that the amplitude of the velocity potential can be written as

$$A = \frac{{P}_{a}}{ρω} ,$$

and thus equation (4) can also be written as a function of acoustic pressure as:

$${F}_{d}^{tr} = ( \frac{{P}_{a}^{2}}{{ρ}_{0} c} ) \frac{{( 2 π )}^{2} {fR}_{0}^{3}}{{c}^{2}} [ {T}_{v} \frac{{δ}_{v}}{{R}_{0}} + {T}_{t} \frac{{δ}_{t}}{{R}_{0}} ] \qquad ( 5 )$$

wherein Pₐ represents pressure amplitude.

Intensity of ultrasound waves is frequently summarized by the spatial peak-temporal average intensity (Iₛₚₜₐ), which is the best measure of the amount of heat delivered to a tissue by ultrasound. Iₛₚₜₐ for a pulsed wave with time varying pressure P(t) can be written as the integral of the pulse power over the pulse length, averaged over the pulse repetition period T_{PRF},

$$I = \frac{1}{{T}_{PRF}} {\int }_{{T}_{p}} \frac{{P ( t )}^{2}}{ρ c} ⅆ t .$$

Hence, the intensity is a linear function of the pulse repetition frequency PRF=1/T_{PRF}. For an undistorted sinusoidal wave with amplitude Pₐ and duty cycle

$$D , {I}_{spta} = ( \frac{{P}_{a}^{2}}{2 {ρ}_{0} c} ) D .$$

The force given by Eq (5) can be seen as linear as a function of acoustic intensity, and a linear function of frequency.

Acoustic streaming in the bulk fluid (without particles) can be described more simply. For a plane wave propagating in an infinite medium, Nightingale provides the expression for the acoustic streaming force (Nightingale and Trahey 2000).

$${F}_{s} = \frac{2 α I}{c} . \qquad ( 6 )$$

wherein α is the absorption coefficient.

The acoustic streaming force is proportional to acoustic intensity, and is a function of the acoustic frequency due to the frequency dependent absorption coefficient α, which is proportional to f² in water, C. M. Daft et al., “Frequency dependence of tissue attenuation measured by acoustic microscopy,” J. Acoust. Soc. Am., 85:2194-2201 (1989)(incorporated herein by reference in its entirety for all purposes). The velocity of the fluid resultant from this force is expressed in the simplest form as Eq. 7 (Nightingale and Trahey 2000).

$${U}_{L} = \frac{α I}{η c} G \qquad ( 7 )$$

wherein U_{L} represents the velocity of the ambient liquid due to acoustic streaming and other nonlinear effects which may develop in the insonified volume and η is the dynamic viscosity of the host fluid.

However, experimental results are highly dependent on the geometric factor G, which describes the boundaries of the medium in which the streaming occurs. Determining G is non-trivial, and the beam and vessel geometry must be carefully considered to avoid significant discrepancies between analytical solutions and experimental measurements of streaming velocity (Shi et al. 2002). Additionally, non-linear propagation produces harmonic frequencies and as a result the streaming velocity imparted in fluid can be substantially greater, H. C. Starritt et al., “An Experimental Investigation of Streaming in Pulsed Diagnostic Ultrasound Beams,” Ultrasound in Med. & Biol., 15:363-73 (1989); J. R. Wu and G. H. Du, “Acoustic Streaming Generated by a Focused Gaussian-Beam and Finite-Amplitude Tonebursts,” Ultrasound in Med. & Biol., 19:167-76 (1993)(each of which is incorporated herein by reference for all purposes).

Acoustic Streaming Versus Radiation Force

The empirically determined relationship between translation velocity and center frequency was approximately a function of frequency squared for data at the lowest pressure studied (a curve fit indicated a power of 2.1, with an R-square value of 0.99, although estimates were tentative since velocity data was only acquired for three frequencies). At the highest pressures studied, curve fitting indicated a power value of 2.3, with an R-square value of 0.94. A further examination of the theory provides some insight into contributions of the forces present on the nanodroplets in an acoustic field.

At small Reynolds numbers, the drag force on a liquid droplet moving in a different immiscible liquid can be written as Eq. 8, L. D. Landau and E. M. Lifshitz, Fluid Mechanics, Oxford: Pergamon (1987)(incorporated herein by reference for all purposes).

$${F}_{drag} = - 2 π η {R}_{0} ( \frac{2 η + 3 \tilde{η}}{η + \tilde{η}} ) ( {U}_{d} - {U}_{L} ) , \qquad ( 8 )$$

wherein U_{d} and U_{L} represent the observed velocity of the droplet due to all forces and the velocity of the ambient liquid due to acoustic streaming and other nonlinear effects which may develop in the insonified volume respectively.

Note that for \tilde{η}→∞, which corresponds to a solid sphere, Eq. (8) turns into the Stokes formula. It is easy to check that in the experiments described, the Reynolds numbers are much smaller than unity for both the acoustical velocity, Vₐ, and the observed values of the translational velocity of the droplets, U_{d}. For example, for I=480 mW/cm², f=10 MHz, R₀=0.1 μm, and U_{d}=200 μm/s, one has
Re(Vₐ)=2R₀Vₐ/v≈0.01, Re(U_{d})=2R₀U_{d}/v≈0.00004.

Therefore Eq. (8) is reasonable approximation. Using this equation, the equation describing the time-averaged translation of the droplet can be written as:

$${m}_{d} \frac{ⅆ {U}_{d}}{ⅆ t} + \frac{2}{3} π {R}_{0}^{3} {ρ}_{0} \frac{ⅆ}{ⅆ t} ( {U}_{d} - {U}_{L} ) = {F}_{drag} + {F}_{rad} \qquad ( 9 )$$

The second term on the left is the added mass force, and F_{rad} is the radiation force which can be estimated by, for example, by Eq. (4). Assuming that U_{L} is constant (over distances traveled by the droplets during measurements) and that U_{d}=0 at t=0, one obtains:

$$\begin{matrix}{U}_{d} = ( {U}_{L} + {U}_{rad} ) ( 1 - exp ( - β t ) ) , \\ where & ( 10 ) \\ \begin{matrix}β = \frac{3 η ( 2 η + 3 \tilde{η} )}{{R}_{0}^{2} ( {ρ}_{0} + 2 \tilde{ρ} ) ( η + \tilde{η} )} , \\ {U}_{rad} = \frac{( η + \tilde{η} ) {F}_{rad}}{2 π ( 2 η + 3 \tilde{η} ) η {R}_{0}} ,\end{matrix} & ( 10 b )\end{matrix}$$

Evaluation of ln 2/β indicates time constants on the order of 0.4 μs for droplets on the order of 1 μm, and less than 5 ns for droplets on the order 0.1 μm for parameters considered here, so droplet velocity approaches streaming velocity within microseconds. The steady-state value of U_{d} tends to the sum U_{L}+U_{rad}.

Let us compare the contributions to Eq. (10) of the streaming velocity, U_{L}, and the radiation force-induced particle velocity, U_{rad}. Using Eq. (4) and experimental relationship δᵥ≈R₀, one has

$$\frac{{U}_{rad}}{{U}_{L}} = \frac{( η + \tilde{η} ) {F}_{rad}}{2 π ( 2 η + 3 \tilde{η} ) η {R}_{0} {U}_{L}} \approx \frac{{ρ}_{0} {\vert A \vert}^{2} {( {kR}_{0} )}^{3}}{η {R}_{0} {U}_{L}} = \frac{ID ω {R}_{0}^{2}}{η {c}^{2} {U}_{L}} . \qquad ( 11 )$$

For I=480 mW/cm², f=10 MHz, and R₀=0.1 μm, one obtains

$$\frac{{U}_{rad}}{{U}_{L}} \approx \frac{{10}^{- 6} m \text{/} s}{{U}_{L}} . \qquad ( 12 )$$

For U_{L}≈100 μm/s, which is on the order of velocities observed in our experiments, the contribution of the radiation force to the motion of the smallest droplets is negligible and hence the motion of the droplet is fully dictated by the acoustic streaming in the bulk liquid. However, the magnitude of U_{rad} increases approximately as a function of R₀², and thus for a droplet with a radius of 1 μm, in fluid with the same streaming velocity (U_{L}≈100 μm/s), the contributions of the radiation and the drag forces can be of the same order and the velocity of the droplet can considerably exceed the velocity of the acoustic streaming. In either case, we estimate that increasing Iₛₚₜₐ will increase both the radiation force and force due to streaming at the same rate. Radiation force increases linearly as a function of f, whereas the streaming force increases nonlinearly as a function of f² (hence the relative contribution of radiation force actually decreases as frequency increases).

In our experiments, although the motion of the droplets could be detected, it was not possible to accurately estimate the diameter of the majority of the droplets due to the limited optical resolution of our system, which we estimate to be on the order of 0.5 micron. Based on the data, we hypothesize that the majority of the droplets were in the size regime where the dominant contribution to the force upon the droplet was due to streaming of the fluid, however, radiation force contributed to the translation as well. In several cases, droplets on the order of one micron were observed, and these droplets did translate to the vessel wall much faster than the majority of nanometer-sized droplets.

### Example 2

### Nanoparticle Oscillation and Displacement

High Speed Photography

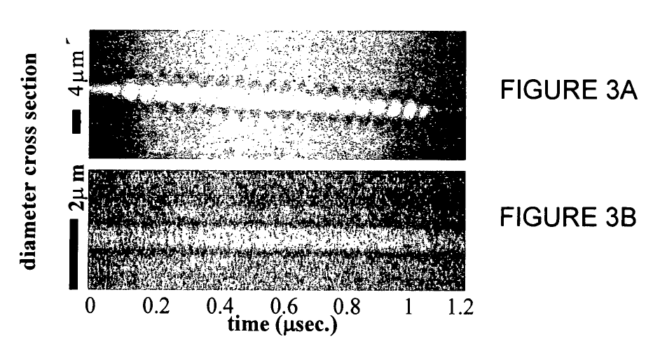

Ultra-high speed photography (10 ns time resolution) of near-micron sized nanoparticles was performed to determine the response of these droplets to ultrasound. The optical system provides two-dimensional “frame” images in addition to a “streak” image, which shows one line-of-sight over time. FIG. 3 combines typical frame and streak images for a lipid-encapsulated, decafluorobutane-filled microbubble and a 100% perfluorohexane nanoparticle. FIG. 3a is an image of the radius-time oscillation of a 2 micron radius microbubble in response to a 180 kPa acoustic pulse at 2.25 MHz. The microbubble oscillates with a maximum expansion 100% greater than its resting diameter. FIG. 3b is an image of the radius-time oscillation of a 450 nanometer radius nanoparticle in response to a 3 MPa acoustic pulse at 10 MHz. The nanoparticle oscillates with a maximum expansion that is barely detectable beyond its resting diameter (less than 5%). The displacement of the microbubble due to acoustic radiation force during the acoustic pulse is on the order of 3 microns, whereas the displacement of the nanoparticle during the acoustic pulse is negligible. The ultrasound parameters used in these examples were selected to maximize the effect on each type of particle. We have previously shown oscillation and displacement of the microbubble to be larger at lower frequencies (Chomas et al. 2001). Nanoparticle oscillation and displacement during a single pulse was not observed for frequencies less than 10 MHz.

Monopole Oscillation of Droplets

Nanoparticles photographed during insonation did not exhibit the large radial oscillations observed with microbubble contrast agents, even at acoustic pressures up to 3 MPa at 10 MHz. These data are in agreement with (Lanza and Wickline 2003), which demonstrated that perfluorocarbon nanoparticles in solution are not readily detectable with clinical frequency ultrasound. It is important to note that Eq. (9) neglects nonlinear effects resulting from the radial oscillation of the droplet by neglecting the time dependence of the radius. Although with the droplets studied, the time variance of the radius is observed to be very small, but it is important to note that for an oscillating particle, additional nonzero mean terms can emerge from the added mass and drag forces. Their contribution to translation can correctly be estimated only by numerical simulations using equations that describe coupled instantaneous radial and translational motions of a liquid droplet, similar to those used in the case of a gas bubble in a strong field. Derivation of such equations is not available in the literature at present, however, other studies have demonstrated that oscillations of highly compressible particles in an acoustic field can result in substantially increased radiation force as the particles undergo larger expansion and contraction near resonance (Dayton et al. 2004).

Although not shown, our experiments demonstrated that nanoparticles experienced a weak attractive force during insonation, producing aggregation suggestive of the presence of secondary radiation force, X. Y. Zheng and R. E. Apfel, “Acoustic Interaction Forces between Two Fluid Spheres in an Acoustic Field,” J. Acoust. Soc. Am., 97:2218-26 (1995); A. A. Doinikov, “Bjerknes forces between two bubbles in a viscous fluid,” J. Acoust. Soc. Am., 106:3305-12 (1999); P. A. Dayton, K. E. Morgan, A. L. S. Klibanov, G. Brandenburger, K. R. Nightingale, and K. W. Ferrara, “A preliminary evaluation of the effects of primary and secondary radiation forces on acoustic contrast agents,” IEEE Trans. Ultrason. Ferroelectr. Freq. Control, 44:1264-77 (1997)(each of which is incorporated herein by reference for all purposes). This attraction was not observed in a dilute solution, but occurred once the nanoparticles were localized against a vessel wall during high intensity insonation.

### Example 3

### Translation of Carrier Particles

We previously developed a model for the displacement produced by radiation forces acting on a contrast agent (P. A. Dayton, J. S. Allen, and K. W. Ferrara, “The magnitude of radiation force on ultrasound contrast agents,” J. Acoust. Soc. Am., 112:2183-92 (2002)), and calculated the translational motion of a microbubble in a fluid during insonation by solving a particle trajectory equation. In addition, we, along with Rychak et al. (J. J. Rychak, et al., “Acoustic radiation force enhances targeted delivery of ultrasound contrast microbubbles: in vitro verification,” IEEE Trans. Ultrason. Ferroelectr. Freq. Control, 52:421-33 (2005)), have shown that acoustic radiation force can enhance the efficiency of targeted imaging and drug delivery with microbubble-based agents by deflecting targeted particles to the endothelium and facilitating bond formation. The radiation force produced on objects with an acoustic impedance several orders of magnitude different from their surrounding medium, such as microbubbles in blood, can produce rapid translation even at acoustic intensities as low as 10 mW/cm². While radiation forces also can deflect liquid and solid particles, the time-averaged intensity required for such effects is substantially greater than for microbubbles.

Data illustrate that for low acoustic pressures, translational velocity is approximately linear as a function of acoustic intensity. This linear dependence observed with acoustic intensity is predicted for streaming of the bulk fluid (Eq. 7), and for the radiation force upon droplets in a viscous heat-conducting fluid (Eq. 5).

Nightingale et al. (2000) have shown that a clinical ultrasound system can produce streaming of bulk fluid in breast cysts with a diameter on the order of millimeters or centimeters. Measurements by Shi et al. (2002) indicate that the translational velocity produced by acoustic streaming in millimeter vessels decreases with decreasing vessel diameter. For many drug delivery applications, acoustic forces must act within capillaries and therefore the effect of these forces in small vessels must be considered.

Nanoparticle Translation

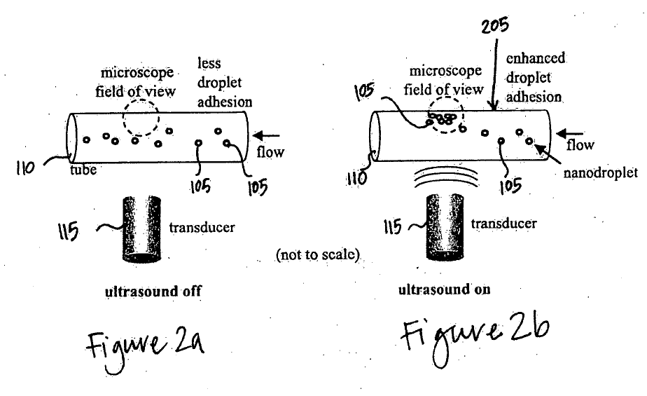

For liquid-filled nanoparticles, such as contrast agents and the carrier particles described herein, we have demonstrated that an acoustic streaming force, produced by insonation can result in rapid translation of the contrast agents and carrier particles. With optimized parameters, the acoustic field produced by a clinical transducer produces translation of the agents away from the acoustic source. In addition, secondary radiation force produces an attractive force between the agents, causing the particles to aggregate. We have thoroughly documented these effects and their relation to acoustic parameters, and in addition, have developed a theoretical model that accurately simulates the displacement of individual particles over an acoustic pulse. A cartoon demonstrating the effect of ultrasound in deflecting the path of carrier particles is shown in FIG. 2. FIG. 2a depicts carrier particles 105 in a tube 110 before the application of ultrasound pressure. Without acoustic streaming and radiation forces, the majority of the contrast agents fail to contact the target site, and therefore do not bind. Following application of ultrasound pressure by the transducer 115, the carrier particles are displaced to the tube wall 205 opposite the transducer 115 (FIG. 2b) by acoustic streaming of the fluid, primary radiation force in some embodiments, and are attracted to each other by secondary radiation force. Ultrasound pushes flowing targeted contrast agents into contact with cells along a vessel wall, where they bind to target receptors.

Ultrasound-induced Nanoparticle Translation

Within a 0.7 mm tube, nanodroplet translation velocity was measured for an Iₛₚₜₐ of 480, 240, and 120 mW/cm², and for 10, 5, 2.25, and 1 MHz. For the first set of experiments, intensity was held constant at 480 mW/cm² to examine the effect of center frequency on translation velocity. Velocity was measured for four different pulse-repetition frequencies, (4, 8, 16, and 32 kHz), while changing the acoustic pressure inversely to pulse repetition frequency (PRF) to observe effects of duty cycle and acoustic pressure. Since the acoustic pressures used for each PRF and each center frequency were different, they are not provided here, however, they can be estimated using the definitions for Iₛₚₜₐ provided in the Introduction.

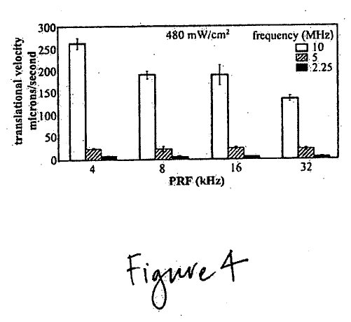

Increasing center frequency while maintaining constant Iₛₚₜₐ, increased translation velocity nonlinearly. FIG. 4 is a graph illustrating translation velocity of insonified nanodroplets in microns/second for 10, 5, and 2.25 MHz. Data are illustrated for four cases of varying PRF. In each case of increasing PRF, acoustic pressure was decreased accordingly to maintain a constant acoustic intensity of 480 mW/cm2. At 10 MHz and 480 mW/cm², nanoparticles translated at velocities on the order of 200 μm/sec, whereas the same intensity at 1 MHz did not produce a measurable translation. Curve fitting to the data indicated that at the highest PRF (corresponding to the lowest acoustic pressures), 32 kHz, velocity increased as a function of frequency to the power of 2.1 (the R-square measure of fit accuracy was 0.99, although the estimate was tentative since velocity data was only acquired for three frequencies). At the lowest PRF studied (corresponding to the highest acoustic pressure), 4 kHz, curve fitting indicated a relationship with frequency to the power of 2.3, with a R-square value of 0.94.

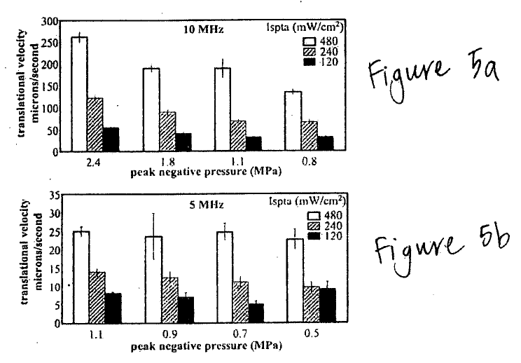

In the second set of experiments, acoustic pressure was held constant and intensity was varied by changing PRF. Data are shown in FIG. 5. FIG. 5 is a graph illustrating translation velocity in microns/second of insonified nanodroplets for 10 MHz (FIG. 5a) and 5 MHz (FIG. 5b), as a function of peak negative pressure. For each decreasing pressure, PRF was increased accordingly to maintain constant intensity. Data are shown for three different values of acoustic intensity.

At 10 MHz, acoustic parameters of 2.4 MPa and 4 kHz, 1.8 MPa and 8 kHz, 1.1 MPa and 16 kHz, and 0.8 MPa and 32 kHz resulted in constant intensity of 480 mW/cm². At these parameters, the 1.8 MPa and 1.1 MPa cases resulted in translation velocities which were not statistically different (p=0.9), however, 2.4 MPa resulted in a slightly higher velocity, and 0.8 MPa resulted in a slightly lower velocity (both p<0.05);

For the 240 and 120 mW/cm² cases, which were achieved by halving or quartering the duty cycle, translational velocities changed less substantially as PRF changed, and in there was no statistical significance between streaming velocities measured in the 1.1 MPa and 0.8 MPa cases (p>0.05).

At 5 MHz, acoustic parameters of 1.1 MPa and 4 kHz, 0.9 MPa and 8 kHz, 0.7 MPa and 16 kHz, and 0.5 MPa and 32 kHz resulted in constant intensity of 480 mW/cm². These parameters resulted in streaming velocities which were not statistically different for the 1.1 MPa, 0.9 MPa, and 0.7 MPa case (p>0.05).

For 240 mW/cm², the streaming velocity was not observed to be statistically different between the 1.1 MPa and 0.9 MPa case, the 0.9 MPa and the 0.7 MPa case, or the 0.7 MPa to 0.5 MPa case (p>0.05). At 120 mW/cm², the streaming velocity was not observed to be statistically different between the 1.1 MPa and 0.9 MPa case, the 0.9 MPa and the 0.7 MPa case (p>0.05).

Nearly all nanodroplets suspended in the solution were observed to translate at approximately the same velocity. Rarely, larger “micro”-droplets, with a diameter on the order of 1 micron were observed. These microdroplets were displaced to the wall of the vessel at a velocity substantially greater than the majority population of droplets in the 300 nm diameter range.

During this experiment, it was observed that once localized along the vessel wall, nanoparticles often aggregated, similar to the clumping of bubbles observed due to secondary radiation force.

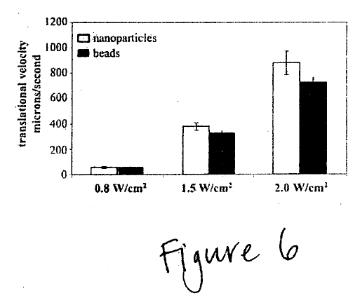

The translational velocity of 100% perfluorohexane nanoparticles and solid beads were evaluated at 10 MHz for Iₛₚₜₐ of 0.8, 1.5, and 2 W/cm². FIG. 6 is a graph illustrating the translation velocity in microns/second for nanodroplets and polystyrene beads insonified at 10 MHz and three acoustic intensities. At the higher intensities the perfluorocarbon droplets are observed to translate slightly faster than the beads. With an Iₛₚₜₐ of 800 mW/cm², the translational velocity of the two formulations of nanoparticles were similar to 500 nm polystyrene beads. At 1.5 W/cm² and 2 W/cm², the droplets moved ˜13% and ˜17% faster than the solid beads, respectively (p<0.05).

Effect of Formulation

During comparison of polystyrene beads with nanodroplets, it was observed that the droplets moved slightly faster than the beads at the highest intensities. Eq. 1 predicts that the contribution of radiation force on the perfluorohexane droplet would be approximately three orders of magnitude greater than the radiation force on a polystyrene particle of the same size (assuming a speed of sound of 2400 m/s and a density of 1050 kg/m³ for polystyrene, D. M. Smith and T. A. Wiggins, “Sound speeds and laser induced damage in polystyrene,” Applied Optics, 11:2680-83 (1972); G. H. Markx et al., “The dielectrophoretic levitation of latex beads, with reference to field-flow fractionation,” J. of Physics D-Applied Physics, 30:2470-77 (1997)(each of which is incorporated herein by reference for all purposes).

Further evaluation of Eq. 1 illustrates that the magnitude of radiation force on the particle increases as both the density and speed of sound in the particle deviate from that of the host medium.

Vessel Size

Translational velocities of the particles measured experimentally were approximately an order of magnitude lower than what would be expected by evaluation of Eq. 7. Shi et al. demonstrated that the effect of the boundary conditions can substantially affect the streaming velocity in a constrained volume, and that streaming velocity decreased for smaller diameter tubes.

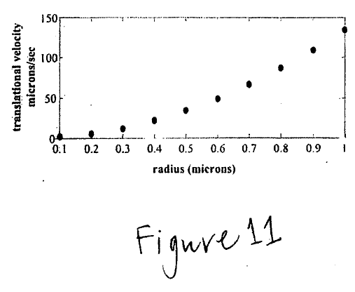

In an in-vivo environment, where nanoparticles would travel through vessels of varying diameters, consideration of particle size would be important to maximize displacement forces. In one embodiment, such vessels include veins, arteries, venules, arterioles, and lymphatics. Vessels also may include capillaries. In capillaries with a typical diameter of 5 microns, the streaming velocity of the fluid may be very small, and the displacement of these particles would be largely due to radiation force. In this case, it would be likely that only the larger particles would experience enough radiation force to cause rapid translation. In larger vessels, the effect of streaming would be more significant, affecting both large and small particles with the fluid. Given that tumor permeability varies greatly with particle diameter, minimizing particle diameter is important, however, the trade-off between extravasation potential and the magnitude of radiation force must be considered. FIG. 11 is a graph illustrating simulations of translational velocity of perfluorohexane nanodroplets from the radiation force component only at 10 MHz and 480 mW/cm² for varying radius, R₀.

These estimated values of nanoparticle translation based on Eq. 11. Simulations based on parameters used in these studies predict that a particle diameter of 500 nm or greater would be required for translation on the order of 10 μm/sec due to radiation force alone.

### Example 4

### Flowing Assay of Localized Delivery of Carrier Particles

Concentration of Targeted Nanoparticles Using Ultrasound in a Flowing System

To evaluate the ability of ultrasound to concentrate targeted nanoparticles in a flowing system, we examined the accumulation of fluorescent biotin-targeted nanoparticles to a 200 μm avidin-coated cellulose microtube, similar to studies described in Zhoa et al. (2004). The sample volume containing flowing nanoparticles was observed for 30 seconds without ultrasound, 30 seconds with ultrasound, and 30 seconds after the ultrasound had been removed. Flow in the tube was maintained at a mean velocity of approximately 9 mm/second. The mean intensity of the tube wall over time was measured offline using MATLAB (Mathworks, Natick, Mass.).

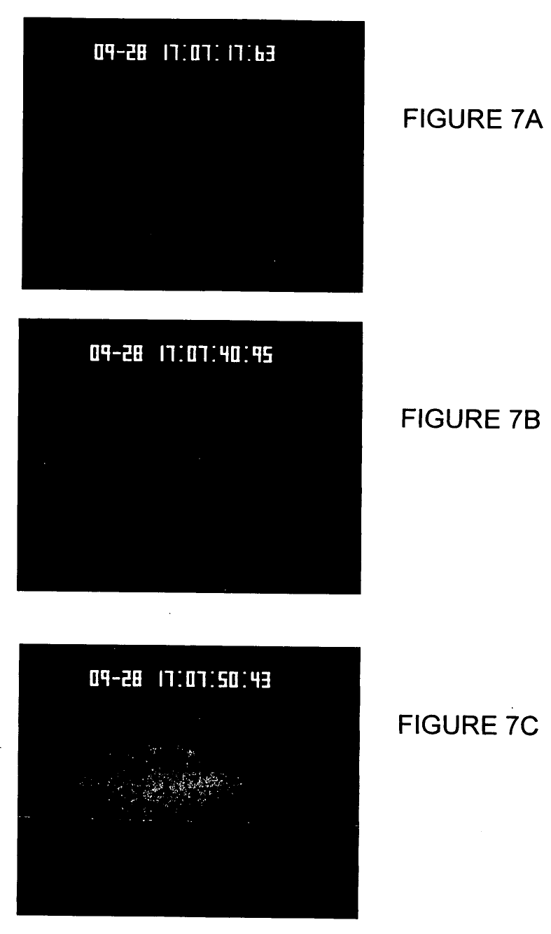

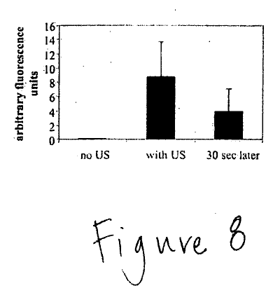

Ultrasound-enhanced retention of biotin-targeted nanoparticles flowing at 9 mm/sec in a 200 μm phantom vessel coated with avidin was evaluated. Sections of the tube were observed with fluorescence microscopy for 30 second periods with, without, and after insonation at 10 MHz and 2.4 W/cm². FIG. 7 shows fluorescence microscopy images illustrating the buildup of fluorescent material from targeted nanodroplets along the wall of a 200 micron vessel before application of ultrasound (FIG. 7a) and during insonation (FIGS. 7b & 7c). Without ultrasound, the fluorescence intensity did not increase above baseline. During insonation, the fluorescence intensity on the tube wall opposite the ultrasound source increased over 100 fold as fluorescent nanoparticles accumulated on the wall surface. After ultrasound was removed, some of the nanoparticles were washed off by the fluid flow, and mean fluorescence intensity decreased by a factor of 2 over 30 seconds. The average fluorescence intensity of 12 tube sections was significantly higher immediately and after 30 seconds after ultrasound application (p<0.05). FIG. 8 is a graph illustrating relative quantitation of the brightness of a phantom vessel through which fluorescent nanoparticles are flowing over 30 second intervals without the application of ultrasound, with ultrasound, and after ultrasound has been removed.

Targeting Specificity

The application of ultrasound was effective in increasing the adhesion of targeted nanoparticles in a flowing model system. In the flowing system, targeted droplet adhesion without insonation was virtually nonexistent. Without ultrasound, few of the droplets approach the proximity required for ligand-receptor interaction with the vessel wall.

### Example 5

### Localized Carrier Particle Drug Delivery Assay

Acoustically-enhanced Delivery of a Fluorescent Dye to Cell Monolayers

Images of the adherent fluorescent vehicle fragments from the dye transfer experiments on cells were acquired by video capture, and the amplitude and spatial extent of the fluorescence at the cell monolayer surface were measured using off-line image processing. Regions of interest (ROI) were chosen based on the beamwidth of 10 MHz insonation, and this ROI was used across all frequencies to normalize for the effect of beamwidth. Fluorescence corresponding to the presence of nanoparticles or due to cell staining was quantified independently by appropriate thresholding, since adherent nanoparticles were brighter than cell membranes which had incorporated DiI.

Application of Ultrasound in Conjunction with Targeted Nanoparticles to Deliver a Fluorescent Dye

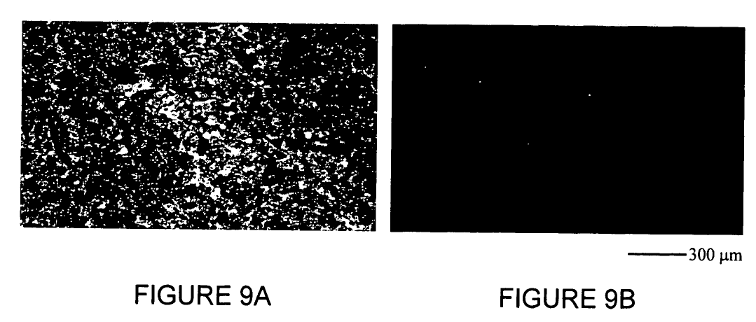

The transfer of the fluorescent dye (DiI) from targeted nanoparticles to a monolayer of PC3 cells was then evaluated within static chambers. FIG. 9 illustrates fluorescence microscopy of PC3 monolayers exposed to targeted nanodroplets containing DiI and ultrasound treatment at 5 MHz and 2.4 W/cm2 for 2 minutes (FIG. 9a), and no ultrasound (FIG. 9b). Two minute exposure to ultrasound with a 5 MHz center frequency and Iₛₚₜₐ of 2400 W/cm², was compared to sham control. Following ultrasound exposure and washing, cells at the acoustic focus were covered with adherent nanoparticles, in contrast to cells outside the acoustic focus which retained few or no nanoparticles. Additionally, after insonation, cells in the acoustic sample volume incorporated the membrane dye carried by the nanoparticles, in contrast to cells outside of the acoustic volume which remained unstained. Without insonation, incubation with nanoparticles (followed by washing) did not result in an increase in fluorescence intensity above baseline.

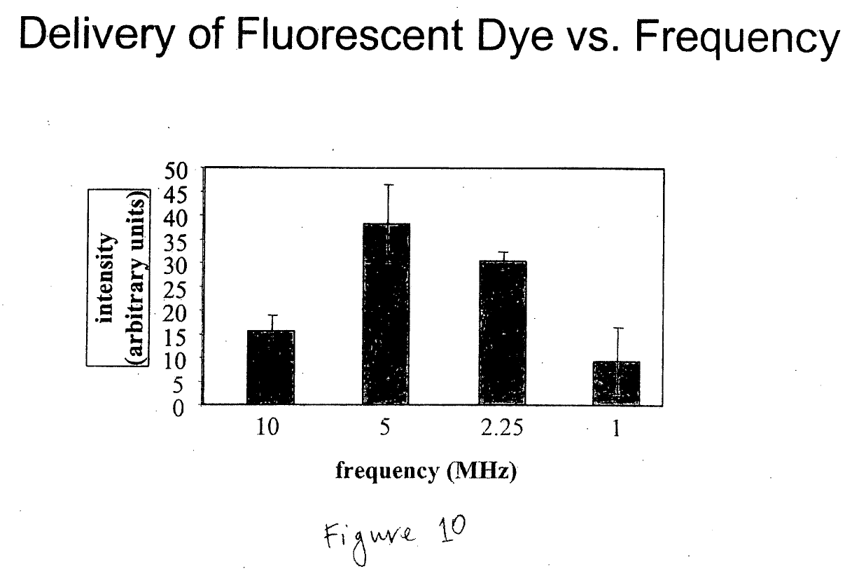

FIG. 10 is a graph illustrating quantitation of brightness of PC3 cells exposed to fluorescent targeted nanodroplets and ultrasound at 10 kHz and 2.4 W/cm2, with center frequencies of 10, 5, 2.25, and 1 MHz. The cellular fluorescence intensity after insonation was estimated for locations within the beam using the insonation parameters listed in Table 3. Data are presented in arbitrary fluorescence units for 10, 5, 2.25, and 1 MHz center frequencies. In these static experiments, transfer of the dye to the cell membrane was significantly greater for a 5 MHz center frequency than for the other frequencies (p<0.05).

Application of Ultrasound in Conjunction with Targeted Nanoparticles to Deliver a Therapeutic Compound

The transfer of a therapeutic compound from targeted nanoparticles to a monolayer of PC3 cells was then evaluated within static chambers, under conditions similar to those described above for transfer of fluorescent dye. FIG. 12 cell toxicity of PC3 monolayers exposed to targeted nanodroplets containing paclitaxel and ultrasound treatment at 5 MHz and 2.4 W/cm2 for 5 minutes, as indicated by Trypan blue staining (FIG. 12a), and treatment with ultrasound only (FIG. 12b). Following ultrasound exposure and washing, cells at the acoustic focus, in the presence of nanoparticles, were killed and stained blue, in contrast to cells outside the acoustic focus which absorbed little to no stain. Ultrasound alone (FIG. 12b) had no therapeutic effect.

FIG. 13 is a graph illustrating quantitation of therapeutic effect on PC3 cells as determined by cell toxicity, according to the conditions described in conjunction with FIG. 12a. Data are presented in quantity of blue pixels, indicating cell toxicity, for paclitaxel-loaded nanoparticles (PAC) loaded with 25 μL/mL with ultrasound, 6 μL/mL with ultrasound, 2 μL/mL with ultrasound, no nanoparticles, and no ultrasound.

Targeting Specificity

The ability to bring nanoparticles into contact with the monolayer was shown to be necessary for efficient transfer of a fluorescent dye (DiI) to the cell monolayer. Without ultrasound, a small number of nanoparticles adhered to the cells on the monolayer; but with application of ultrasound, both the concentration of the nanoparticles on the cell surface and the corresponding transfer of the fluorescent nanoparticle contents to the cells increased substantially. Although it initially appeared that 5 MHz may have had the most significant effect on the concentration of nanoparticles, none of the data was statistically different due to small sample numbers for each parameter set.

Safety

Ultrasound effectively increased the local concentration of targeted nanoparticles, both in static solution, and in flow up to 9 mm/sec in a 200 micron vessel, however, the acoustic intensities utilized were over the Iₛₚₜₐ limit of 720 mW/cm² for ultrasonic imaging. The safety of this technique in vivo has not been established, although our results with trypan blue suggest no cellular toxicity from the ultrasound per se.

Limitations of These Studies

It was observed that measurements of the nanodroplet translation velocity were very sensitive to the location at which the measurements were taken inside the tube. However, once the optical and acoustical focus was chosen, translation velocity measurements had a very small standard deviation. Small changes in the tube position relative to the optical and acoustical focus, which may have occurred over time may have influenced the translation velocity between data sets, although all measurements within one set appeared consistent. We note that although most of the standard deviation bars in FIGS. 4-6 appear very small, they do not convey this additional error margin which occurred between data sets.

Several limitations of the theory in this manuscript should be noted. With the diameters and frequencies tested, the nanodroplets do not fully meet the criteria for weak dissipation required for Eq 4. of R₀>>δᵥ or R₀>>\tilde{δ}ᵥ, since in our case these terms are closer to the same order of magnitude. A new theoretical development for insonation of nanometer droplets will be required to more accurately characterize the effect of acoustic radiation forces on these particles.

Another factor confounding this analysis is the presence of significant non-linear wave propagation for the acoustic pressures and frequencies studied which is not accounted for with Eq. 7, which will substantially increase the streaming velocity of a fluid (Starritt et al. 1989). At the highest acoustic pressure studied, which was applied at 10 MHz, the nanoparticles translated faster than the assumed linear relation with acoustic intensity would predict. Non-linear propagation at high intensities may have increased the translational velocity in this circumstance.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for enhancing localized delivery of a compound, comprising:
administering into a fluid carrier particles comprising the compound;
exposing said fluid and said carrier particles within said fluid to an ultrasound wave at a first center frequency and pressure combination, wherein said first center frequency and pressure combination generates acoustic streaming of said fluid; and
wherein said acoustic streaming enhances localized delivery of said compound.

2. The method of claim 1, wherein said first center frequency and pressure combination produces a spatial peak-temporal average intensity from 200 mW/cm² to 8 W/cm².

3. The method of claim 1, wherein said acoustic streaming enhances localized delivery of said compound by concentrating said carrier particles.

4. The method of claim 1, wherein said acoustic streaming enhances localized delivery of said compound by altering a translational velocity of said carrier particles.

5. The method of claim 1, wherein the diameter of said carrier particles is less than about 1 micron.

6. The method of claim 1, wherein the diameter of said carrier particles is less than about 750 nm.

7. The method of claim 1, wherein the diameter of said carrier particles is less than about 500 nm.

8. The method of claim 1, wherein said acoustic streaming enhances localized delivery of said compound by promoting binding of said carrier particles to a localized target site.

9. The method of claim 1, wherein said carrier particles comprise a liquid core.

10. The method of claim 1, wherein said carrier particles comprise a lipid membrane.

11. The method of claim 10, wherein said lipid membrane is caused to fuse with a cell membrane.

12. The method of claim 1, wherein said carrier particles experience an ultrasonic radiation force.

13. The method of claim 1, wherein said fluid is within a cavity.

14. The method of claim 1, wherein said fluid is within a vessel.

15. The method of claim 14, wherein said vessel is selected from the group consisting of a vein, an artery, a venule, an arteriole, and a lymphatic.

16. The method of claim 1, further comprising imaging said carrier particles.

17. The method of claim 16, wherein said imaging is optical.

18. The method of claim 16, wherein said imaging is magnetic resonance imaging.

19. The method of claim 16, wherein said imaging is ultrasonic imaging.

20. The method of claim 1, wherein said carrier particles comprise a perfluorocarbon liquid.

21. The method of claim 20, wherein said perfluorocarbon liquid has a density between 1.5 and 2.1 g/cm³ at 25° C. at 1 atm.

22. The method of claim 1, wherein said carrier particles comprise a perfluorocarbon which undergoes a liquid to a gas phase transition between 25° C. and 42° C. at 1 atm.

23. The method of claim 1, wherein said carrier particles comprise an oil.

24. The method of claim 23, wherein said oil has a density ranging from 0.7 to 1.7 g/ml at 25° C. at 1 atm.

25. The method of claim 1, wherein said carrier particles further comprise a targeting moiety.

26. The method of claim 25, wherein said targeting moiety is selected from the group consisting of an antibody, an antibody fragment, an aptamer, a carbohydrate, a polysaccharide, a polypeptide, a peptidomimetic, a nucleic acid, and a small organic molecule.

27. The method of claim 26, wherein said polypeptide is a peptidic adhesion ligand.

28. The method of claim 1, wherein said compound is a therapeutic compound.

29. The method of claim 28, wherein said compound inhibits cell division.

30. The method of claim 29, wherein said compound is paclitaxel.

31. The method of claim 1, wherein said compound is a diagnostic compound.

32. The method of claim 1, wherein said compound increases vessel permeability.

33. The method of claim 1, wherein said carrier particles are selectively displaced independent of blood cells.

34. The method of claim 1, further comprising generating an ultrasonic image of a localized delivery region.

35. The method of claim 1, further comprising sonoporating a cell or a tissue at a localized delivery site.

36. The method of claim 35, wherein at least a component of said sonoporating arises from an interaction between said carrier particles and said ultrasound wave.

37. The method of claim 36, wherein said interaction produces a local increase in a cell membrane permeability.

38. The method of claim 1, further comprising vasoporating an endothelium at a localized delivery site.

39. The method of claim 38, wherein at least a component of said vasoporation arises from an interaction between said carrier particles and said ultrasound wave.

40. The method of claim 39, wherein said interaction produces a local increase in a vessel permeability.
